(19) United States
(12) Patent Application Publication
(10) Pub. No.: US 2025/0034107 A1
(43) Pub. Date: Jan. 30, 2025

(54) SUBSTITUTED 2-(2-(2,6-DIOXOPIPERIDIN-3-YL)-1- OXOISOINDOLIN-5-YL) ACETAMIDE ANALOGS AS MODULATORS OF GSPT1 AND/OR IKZF1 PROTEIN

(71) Applicant: St. Jude Children's Research Hospital, Inc., Memphis, TN (US)

(72) Inventors: Gisele A. NISHIGUCHI, Memphis, TN (US); Zoran RANKOVIC, Memphis, TN (US); Fatemeh KERAMATNIA, Memphis, TN (US)

(21) Appl. No.: 18/712,960

(22) PCT Filed: Nov. 17, 2022
(86) PCT No.: PCT/US2022/050325

§ 371 Date: May 23, 2024

Related U.S. Application Data
(60) Provisional application No. 63/282,512, filed on Nov. 23, 2021.

(51) Int. Cl. C07D 401/04; A61K 31/454; A61K 31/5377

(52) U.S. Cl. CPC C07D 401/04; A61K 31/454; A61K 31/5377

(57) ABSTRACT

In one aspect, the disclosure relates to substituted 2-(2-(2,6-dioxopiperidin-3-yl)-1-oxoisoindolin-5-yl)acetamide analogs that useful as modulators of GSPT1 and/or IKZF1 activity, methods of making same, pharmaceutical compositions comprising same, and methods of treating various clinical conditions and disorders using same, e.g., a disorder of uncontrolled cellular proliferation, such as a cancer, which may be associated with GSPT1 and/or IKZF1 protein dysfunction. This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/282,512, filed Nov. 23, 2021, which is incorporated herein by reference in its entirety.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). A tremendous demand exists for new methods, treatments, and compositions that can be used to treat patients with cancer.

Protein degradation plays a role in various cellular functions, i.e., the concentrations of regulatory proteins are adjusted through degradation into small peptides to maintain health and productivity of the cells. Cereblon is a protein that forms an E3 ubiquitin ligase complex, which ubiquitinates various other proteins. Specifically targeting protein degradation offers a tantalizing prospect of targeting currently undruggable oncoproteins such as transcription factors and chimeric fusion oncoproteins.

Despite advances in research directed to clinical amelioration of cancer, there is still a scarcity of compounds that are both potent, efficacious, and selective targeted modulators of protein degradation, e.g., potent and selective modulation of GSPT1 and IKZF1 degradation. These needs and other needs are satisfied by the present disclosure.

### SUMMARY

In accordance with the purpose(s) of the disclosure, as embodied and broadly described herein, the disclosure, in one aspect, relates to substituted 2-(2-(2,6-dioxopiperidin-3-yl)-1-oxoisoindolin-5-yl)acetamide analogs that are useful as modulators of GSPT1 and/or IKZF1 protein, methods of making same, pharmaceutical compositions comprising same, and methods of treating various clinical conditions and disorders using same, e.g., a disorder of uncontrolled cellular proliferation, such as a cancer, which may be associated with a GSPT1 and/or IKZF1 protein dysfunction.

Disclosed are compounds having a structure represented by a formula:

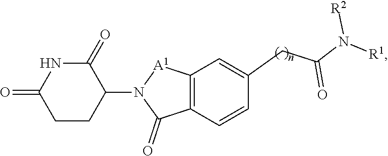

wherein each of A¹ is selected from —(C═O)— and —(CH₂)—; wherein n is selected from 1, 2, and 3; wherein R¹ is selected from —(C1-C3 alkanediyl)-phenyl and —(C1-C3 alkanediyl)-naphthyl; and wherein R¹ is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰; wherein -A¹⁰ is selected from substituted naphthyl, substituted phenyl, substituted cycloalkyl, and substituted heterocycloalkyl; and wherein -A²⁰- is selected from —(CH₂)ₓ—, —(O—CH₂)ₓ—, —(NH—CH₂)ₓ—, —(CH₂—O)ₓ—, and —(CH₂—NH)ₓ—; wherein x is selected from 0, 1, 2, and 3; wherein R² is selected from hydrogen, substituted C1-C10 alkyl, and substituted C1-C10 heteroalkyl; or a pharmaceutically acceptable salt thereof.

Also disclosed are pharmaceutical compositions comprising a therapeutically effective amount of one or more disclosed compounds, or pharmaceutically acceptable salt thereof, and a pharmaceutically acceptable carrier.

Also disclosed are methods for the treatment of a disorder of uncontrolled cellular proliferation in a mammal comprising the step of administering to the mammal a therapeutically effective amount of at least one disclosed compound or pharmaceutically acceptable salt thereof, or at least one disclosed pharmaceutical composition.

Also disclosed are methods for modulating of cereblon activity in a mammal comprising the step of administering to the mammal a therapeutically effective amount of at least one disclosed compound or pharmaceutically acceptable salt thereof, or at least one disclosed pharmaceutical composition.

Also disclosed are methods for modulating of GSPT1 activity in a mammal comprising the step of administering to the mammal a therapeutically effective amount of at least one disclosed compound or pharmaceutically acceptable salt thereof, or at least one disclosed pharmaceutical composition.

Also disclosed are methods for modulating of IKZF1 activity in a mammal comprising the step of administering to the mammal a therapeutically effective amount of at least one disclosed compound or pharmaceutically acceptable salt thereof, or at least one disclosed pharmaceutical composition.

Also disclosed are methods for modulating of cereblon activity in at least one cell, comprising the step of contacting the at least one cell with an effective amount of at least one disclosed compound, or a pharmaceutically acceptable salt thereof; or at least one disclosed pharmaceutical composition.

Also disclosed are methods for modulating of GSPT1 activity in at least one cell, comprising the step of contacting the at least one cell with an effective amount of at least one disclosed compound, or a pharmaceutically acceptable salt thereof; or at least one disclosed pharmaceutical composition.

Also disclosed are methods for modulating of IKZF1 activity in at least one cell, comprising the step of contacting the at least one cell with an effective amount of at least one disclosed compound, or a pharmaceutically acceptable salt thereof; or at least one disclosed pharmaceutical composition.

Also disclosed are uses of a disclosed compound, or a pharmaceutically acceptable salt thereof; a disclosed product of making, or a pharmaceutically acceptable salt thereof; or a disclosed pharmaceutical composition.

Also disclosed are uses of a disclosed compound, or a pharmaceutically acceptable salt thereof, in the manufacture of a medicament for the treatment of a disorder associated with a cereblon protein dysfunction in a mammal.

Also disclosed are uses of a disclosed compound, or a pharmaceutically acceptable salt thereof, in the manufacture of a medicament for the treatment of a disorder associated with a GSPT1 protein dysfunction in a mammal.

Also disclosed are uses of a disclosed compound, or a pharmaceutically acceptable salt thereof, in the manufacture of a medicament for the treatment of a disorder associated with a IKZF1 protein dysfunction in a mammal.

Also disclosed are uses of a disclosed compound, or a pharmaceutically acceptable salt thereof, in the manufacture of a medicament for the treatment of a disorder associated with a cellular proliferation dysfunction in a mammal, e.g., to inhibit cellular proliferation in a cancer cell, in a mammal comprising combining at least one disclosed compound, or a pharmaceutically acceptable salt thereof with a pharmaceutically acceptable carrier or diluent.

Also disclosed are methods for the manufacture of a medicament to modulate the cereblon protein in a mammal comprising combining at least one disclosed compound, or a pharmaceutically acceptable salt thereof with a pharmaceutically acceptable carrier or diluent.

Also disclosed are methods for the manufacture of a medicament to modulate the GSPT1 protein in a mammal comprising combining at least one disclosed compound, or a pharmaceutically acceptable salt thereof with a pharmaceutically acceptable carrier or diluent.

Also disclosed are methods for the manufacture of a medicament to modulate the IKZF1 protein in a mammal comprising combining at least one disclosed compound, or a pharmaceutically acceptable salt thereof with a pharmaceutically acceptable carrier or diluent.

Also disclosed are methods for the manufacture of a medicament to inhibit cellular proliferation, e.g., to inhibit cellular proliferation in a cancer cell, in a mammal comprising combining at least one disclosed compound, or a pharmaceutically acceptable salt thereof with a pharmaceutically acceptable carrier or diluent.

Also disclosed are kits comprising at least one disclosed compound, or a pharmaceutically acceptable salt thereof; or at least one disclosed pharmaceutical composition; and one or more of: (a) at least one agent known to increase cereblon activity; (b) at least one agent known to decrease cereblon activity; (c) at least one agent known to increase GSPT1 activity; (d) at least one agent known to decrease GSPT1 activity; (e) at least one agent known to increase IKZF1 activity; (f) at least one agent known to decrease IKZF1 activity; (g) at least one agent known to increase cellular proliferation; (h) at least one agent known to decrease cellular proliferation; (i) at least one agent known to treat a disorder associated with cereblon activity; (j) at least one agent known to treat a disorder associated with GSPT1 activity; (k) at least one agent known to treat a disorder associated with IKZF1 activity; (l) at least one agent known to treat a disorder of uncontrolled cellular proliferation; and/or (m) instructions for treating a disorder of uncontrolled cellular proliferation.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure.

Moreover, in the drawings, like reference numerals designate corresponding parts throughout the several views.

FIG. 1A shows a schematic representation of disclosed compounds comprising certain chemical features associated with the disclosed compounds.

FIG. 1B shows a schematic representation of a disclosed compound interacting with a target protein, i.e., protein of interest, and a cereblon protein, resulting in stabilization of a protein interface between the target protein and cereblon. Also shown are proteins DDB1, RBX1, and E2 forming a complex with cereblon. In the figure, E2 is involved with tagging the protein of interest for degradation by ligation of one or more ubiquitin residues to the target protein.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings.

Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a compound,” “a substituent group,” or “a cancer,” including, but not limited to, two or more such compounds, substituent groups, or cancers, including combinations of compounds, substituent groups, or cancers.

Reference to “a/an” chemical compound, protein, and antibody each refers to one or more molecules of the chemical compound, protein, and antibody rather than being limited to a single molecule of the chemical compound, protein, and antibody. Furthermore, the one or more molecules may or may not be identical, so long as they fall under the category of the chemical compound, protein, and antibody. Thus, for example, “an” antibody is interpreted to include one or more antibody molecules of the antibody, where the antibody molecules may or may not be identical (e.g., different isotypes and/or different antigen binding sites as may be found in a polyclonal antibody).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “cereblon” and “CRBN” can be used interchangeably, and refer to a protein encoded by the CRBN gene in humans with a cytogenetic location of 3p26.2 and a molecular location of base pairs 3,148,489 to 3,179,716 on chromosome 3 (UCSC Genome Browser on Human December 2013 (GRCh38/hg38) Assembly). The gene structure in humans comprises 11 exons. CRBN is a substrate recognition component of a DCX (DDB1-CUL4-X-box) E3 protein ligase complex that mediates the ubiquitination and subsequent proteasomal degradation of target proteins. The DCX (DDB1-CUL4-X-box) E3 protein ligase complex is composed at least of CRBN, CUL4A, DDB1, and RBX1. The CRBN protein has two isoforms produced by alternative splicing: Isoform 1 has 442 amino acids and a molecular weight of 50,546 Da; and Isoform 2 has 441 amino acids and a molecular weight of 50,475 Da.

As used herein, “GSPT1” refers to eukaryotic peptide chain release factor GTP-binding subunit ERF3A, a protein believed to be involved translation termination in response to the termination codons UAA, UAG and UGA, is believed to be stimulated by ETF1, and is likely involved in mammalian cell growth. In humans, GSPT1 has a cytogenetic location of 16p13.1 and a molecular location of base pairs 11,868,128-11,916,654 on chromosome 16 (GRCh38/hg38). The gene structure is humans comprises 16 exons. GSPT1 is believed to be largely a cytosolic protein. GSPT1 has also been referred to as ERF3a, ERF3A, ETF3A, GST1, eukaryotic Peptide Chain Release Factor Subunit 3a, G1 To S Phase Transition Protein 1 Homolog, 551G9.2, and Eukaryotic Release Factor 3a.

As used herein, “IKZF1” refers to Ikaros family zinc finger 1, a member of a family of hematopoietic zinc finger transcription factors and it is believed to be involved in gene expression via chromatin remodeling. In humans it has a cytogenetic location of 7p12.2 and a molecular location of base pairs 50,303,453-50,405,101 on chromosome 7 (GRCh33/hg38).

The gene structure is humans comprises seven translated exons. IKZF1 is believed to be distributed in the cytosol and nucleus. The expression of this protein is restricted to the fetal and adult hemo-lymphopoietic system, and it functions as a regulator of lymphocyte differentiation. Several alternatively spliced transcript variants encoding different isoforms have been described for this gene. IKZF1 has also been referred to as IKAROS, Hs.54452, PPP1R92, ZNFN1A1, LyF-1, LYF1, IK1, Protein Phosphatase 1—Regulatory Subunit 92, Lymphoid Transcription Factor LyF-1, Hlk-1, CLL-Associated Antigen KVV-6, Ikaros Family Zinc Finger Protein 1, and Zinc Finger Protein, Subfamily 1A, 1 (Ikaros).

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g., by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like.

Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-r interactions, anion-r interactions, polar r-interactions, and hydrophobic effects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a disorder of uncontrolled cellular, e.g., a cancer such as acute leukemia or a medulloblastoma. The effect can be therapeutic in terms of a partial or complete cure of a disease, condition, symptom or adverse effect attributed to the disease, disorder, or condition. The term “treatment” as used herein can include any treatment of a disorder of uncontrolled cellular, e.g., a cancer such as acute leukemia or a medulloblastoma, in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, nomenclature for compounds, including organic compounds, can be given using common names, IUPAC, IUBMB, or CAS recommendations for nomenclature.

When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Reference to “a” chemical compound refers to one or more molecules of the chemical compound rather than being limited to a single molecule of the chemical compound. Furthermore, the one or more molecules may or may not be identical, so long as they fall under the category of the chemical compound. Thus, for example, “a” chemical compound is interpreted to include one or more molecules of the chemical, where the molecules may or may not be identical (e.g., different isotopic ratios, enantiomers, and the like).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, the term “effective amount” refers to an amount that is sufficient to achieve the desired modification of a physical property of the composition or material. For example, an “effective amount” of a disclosed compound or pharmaceutical composition refers to an amount that is sufficient to achieve the desired degree of modulation of a target, e.g., modulation of cereblon protein, or a desired amelioration or improvement in a clinical condition, e.g., remission of a cancer. The specific level in terms of amount, e.g., milligrams, or concentration, e.g., micromolar, of a disclosed compound or pharmaceutical composition required as an effective amount will depend upon a variety of factors the route of administration or contacting with the target, the severity of a clinical condition, the desired degree of modulation, and the like.

As used herein, the terms “optional” or “optionally” means that the subsequently described event or circumstance can or cannot occur, and that the description includes instances where said event or circumstance occurs and instances where it does not.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic.

The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g., fluorine, chlorine, bromine, or iodine. The term “polyhaloalkyl” specifically refers to an alkyl group that is independently substituted with two or more halides, i.e., each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as -OA¹ where A¹ is alkyl or cycloalkyl as defined above. “Alkoxy” also includes polymers of alkoxy groups as just described; that is, an alkoxy can be a polyether such as -OA¹-OA² or -OA¹-(OA²)ₐ-OA³, where “a” is an integer of from 1 to 200 and A¹, A², and A³ are alkyl and/or cycloalkyl groups.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “hydroxyl” or “hydroxy” as used herein is represented by the formula —OH.

The term “thiol” as used herein is represented by the formula —SH.

The term “azide” or “azido” as used herein is represented by the formula —N₃.

The term “nitro” as used herein is represented by the formula —NO₂.

The term “nitrile” or “cyano” as used herein is represented by the formula —CN.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, 0, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “carbonyl” as used herein is represented by the formula —C(O)—.

Throughout this specification “C(O)” or C═O is a short hand notation for a carbonyl group.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl.

Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “heterocycle” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

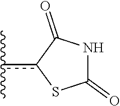

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present disclosure unless it is indicated to the contrary elsewhere herein.

The term “stable,” as used herein, refers to compounds that are not substantially altered when subjected to conditions to allow for their production, detection, and, in certain aspects, their recovery, purification, and use for one or more of the purposes disclosed herein.

Compounds described herein can contain one or more double bonds and, thus, potentially give rise to cis/trans (E/Z) isomers, as well as other conformational isomers. Unless stated to the contrary, the disclosure includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present disclosure includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, sulfur, fluorine and chlorine, such as ²H, ³H, ¹³C, ¹⁴C ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this disclosure. Certain isotopically-labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present disclosure and prodrugs thereof can generally be prepared by carrying out the procedures below, by substituting a readily available isotopically labeled reagent for a non-isotopically labeled reagent.

The compounds described in the disclosure can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the disclosure to form solvates and hydrates. Unless stated to the contrary, the disclosure includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g., “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

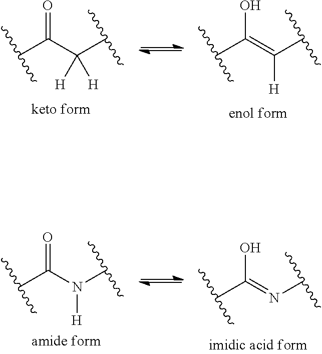 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the disclosure includes all such possible tautomers.

It is known that chemical substances form solids which are present in different states of order which are termed polymorphic forms or modifications. The different modifications of a polymorphic substance can differ greatly in their physical properties. The compounds according to the disclosure can be present in different polymorphic forms, with it being possible for particular modifications to be metastable. Unless stated to the contrary, the disclosure includes all such possible polymorphic forms.

In some aspects, a structure of a compound can be represented by a formula:

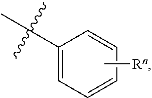

which is understood to be equivalent to a formula:

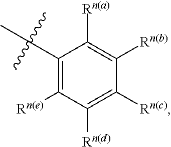

wherein n is typically an integer. That is, Rⁿ is understood to represent five independent substituents, R^{n(a)}, R^{n(b)}, R^{n(c)}, R^{n(d)} and R^{n(e)}. By “independent substituents,” it is meant that each R substituent can be independently defined. For example, if in one instance R^{n(a)} is halogen, then R^{n(b)} is not necessarily halogen in that instance.

Unless otherwise specified, temperatures referred to herein are based on atmospheric pressure (i.e., one atmosphere).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific embodiment or combination of embodiments of the methods of the disclosure.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Abbreviations used herein throughout: ADME, absorption, distribution, metabolism, excretion; AL, acute leukemia; AlogP, lipophilicity; CK1α, casein kinase 1α; CL, clearance; CRBN, cereblon; F, bioavailability; GSPT1, G1 to S phase transition 1; HBA, hydrogen-bond acceptors; HBD, hydrogen-bond donors; IMiDs, immunomodulatory drugs; IKZF, Ikaros family zinc finger; IV, intravenous; LCMS, liquid chromatography mass spectrometry; MB, medulloblastoma; MG, molecular glue; MW, molecular weight; PPB, plasma protein binding; PK, pharmacokinetics; PROTAC, proteolysis targeting chimera(s); PSA, polar surface area; SBDD, structure-based drug design; SAR, structure-activity relationships; TMT, tandem mass tag; and TPD, targeted protein degradation.

Described herein are substituted 2-(2-(2,6-dioxopiperidin-3-yl)-1-oxoisoindolin-5-yl)acetamide analogs that have therapeutic or clinical utility as that are useful as modulators of GSPT1 and/or IKZF1 protein. Also described herein are methods of synthesizing the substituted 2-(2-(2,6-dioxopiperidin-3-yl)-1-oxoisoindolin-5-yl)acetamide analogs. Also described herein are methods of administering the substituted 2-(2-(2,6-dioxopiperidin-3-yl)-1-oxoisoindolin-5-yl)acetamide analogs to a subject in need thereof. In some aspects, the subject can have a disorder of uncontrolled cellular proliferation, e.g., a cancer. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### B. BIOLOGICAL CONTEXT

Targeted Protein Degradation (TPD) is a novel chemical biology approach with potential profound effects on fundamental biology and drug discovery research by uncovering opportunities towards drugging undruggable targets (see Refs. 1-2). The TPD paradigm includes two main approaches of differing molecular design that generate small molecules with a similar proteasome dependent mechanism of action, namely Proteolysis Targeting Chimeras (PROTACs; see Refs. 3-4) and Molecular Glues (MGs; see Refs. 5-8). MGs are small molecules capable of binding to an E3 ligase and altering its surface and specificity, leading to the recruitment, ubiquitination, and subsequent degradation of substrates that are normally not targeted by the ligase (neosubstrates). Recognition of the neosubstrate is governed by a protein-ligase surface interaction (a structural degron motif) and does not require a ligandable pocket. This provides a revolutionary opportunity to degrade hitherto undruggable targets, such as fusion oncoproteins and transcription factors (see Refs. 9-10) Immunomodulatory drugs (IMiDs), thalidomide and its close analogs pomalidomide and lenalidomide, are the “original” molecular glues providing the mechanistic and clinical validation of this approach (see Refs. 7 and 11)

Interestingly, despite high molecular structural similarity, IMiDs display different protein degradation profiles. Both lenalidomide and pomalidomide degrade the transcription factors IKZF1/3 but only lenalidomide induces degradation of CSNK1A1 (CK1a), illustrating how a small change in molecular structure can significantly alter the specificity for the neosubstrate (see Ref. 12). Moreover, diversification around the IMiDs scaffold has been shown to influence the potency and kinetics of neosubstrate degradation (exemplified by CC-220, which is 10-fold more potent in cells than lenalidomide; see Ref. 13) or specificity, leading to the discovery of novel neosubstrates, as demonstrated by the GSPT1 (G1 to S phase transition 1) degrader CC-885 developed by Celgene (see Ref. 14). These chemical modifications lead to considerable changes in cellular responses, creating new clinical translation opportunities.

For IMiDs and closely related analogs, an expanding number of neosubstrates containing the common C2H2 zinc finger recognition degron motif have been discovered (IKZF2/4, SALL4, RNF166, ZFP91, ZNF692, ZNF276, ZNF653 and ZNF827; see Refs.11 and 15). Each IMiD was shown to display distinct patterns of substrate specificity, supporting the notion that neosubstrate diversity can be modulated by structural alterations of the ligand and is not limited to traditionally known targets. It also suggests that achieving selective protein degradation is a challenge and that understanding the structural basis of how ligand modification alters the interaction of the neosubstrate at the cereblon (CRBN) interface is important (Ref. 16). Several recently reported studies showed how simple structural modifications can result in an unexpected conversion of a PROTAC into a GSPT1 molecular glue degrader (see Refs. 17-18).

In high-risk cancers like childhood acute leukemia (AL) and medulloblastoma (MB), aberrant activation, dysregulation and/or mutation of C2H2 zinc finger transcription factors are prevalent, with limited targeted treatments. Representative examples include ZNF384 fusion oncoproteins observed in lineage ambiguous acute leukemia,19 IKZF1 mutations in acute lymphoblastic leukemia (ALL; see Ref. 20), deregulated MECOM in high-risk acute myeloid leukemia (AML) and enhancer-hijacking dependent activation of GFI1, GFI1B, and PRDM6 in high-risk group 3 and group 4 MB subgroups (see Refs. 21-24).

Small molecule degraders, often referred to as molecular glues, offer the tantalizing prospect of targeting currently undruggable oncoproteins such as transcription factors and chimeric fusion oncoproteins. The present disclosure relates to methods and uses of small molecule degraders that modulate CRBN protein and show high anti-proliferative activity.

### C. COMPOUNDS

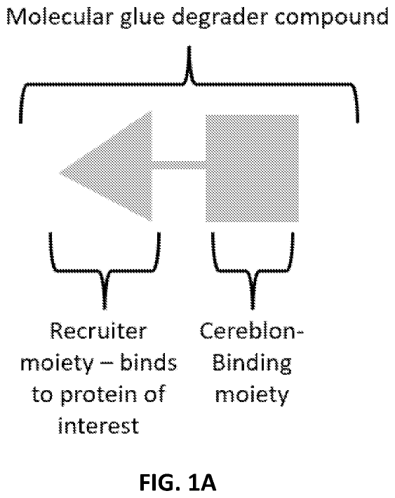

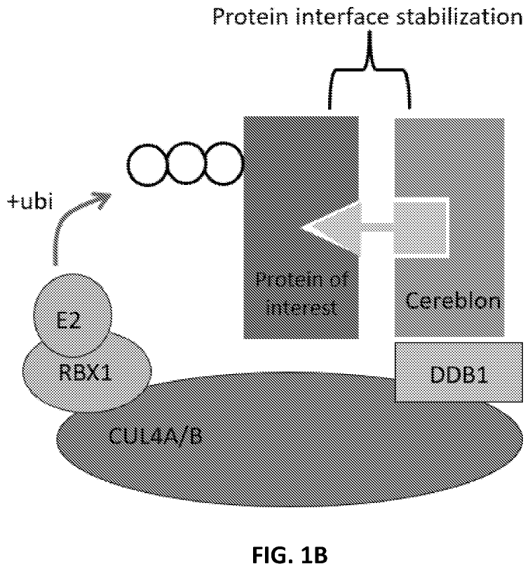

In one aspect, the disclosure relates to potent modulators of protein degradation, comprising, in part, a moiety or substructure that binds or interacts with cereblon and a moiety or substructure that binds or interacts with a target protein(s). Without wishing to be bound by a particular theory, it is believed that the disclosed compounds have chemical features for CRBN engagement while maximizing the 3-dimensionality of chemical diversity displayed at the CRBN substrate binding surface. The disclosed compounds provide compounds useful for providing new approaches to target previously undruggable target proteins, and therefor, provide new opportunities for treating a variety of disorders, including cancer. Without wishing to be bound by a particular theory, the disclosed compounds, as shown in a schematic representation in FIG. 1A, comprise chemical features or moieties designed to interact with a target protein of therapeutic interest and cereblon protein. That is, the disclosed compounds can act as molecular glues via simultaneous interaction between the target protein and cereblon, and in doing so, stabilize a protein interface between the target protein and cereblon. A disclosed compound interacting with both a target protein, i.e., protein of interest such as GSPT1 and/or IKZF1, and cereblon protein is shown schematically in FIG. 1B.

In one aspect, the present invention pertains to compounds having a structure represented by a formula:

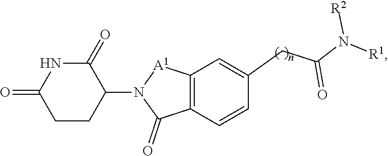

wherein each of A¹ is selected from —(C═O)— and —(CH₂)—; wherein n is selected from 1, 2, and 3; wherein R¹ is selected from —(C1-C3 alkanediyl)-phenyl and —(C1-C3 alkanediyl)-naphthyl; and wherein R¹ is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰; wherein -A¹⁰ is selected from substituted naphthyl, substituted phenyl, substituted cycloalkyl, and substituted heterocycloalkyl; and wherein -A²⁰- is selected from —(CH₂)ₓ—, —(O—CH₂)ₓ—, —(NH—CH₂)ₓ—, —(CH₂—O)ₓ—, and —(CH₂—NH)ₓ—; wherein x is selected from 0, 1, 2, and 3; wherein R² is selected from hydrogen, substituted C1-C10 alkyl, and substituted C1-C10 heteroalkyl; or a pharmaceutically acceptable salt thereof.

In various aspects, the disclosed compounds are potent modulators of GSPT1 and/or IKZF1 protein degradation. Without wishing to be bound by a particular theory, it is believed that the disclosed compounds act as modulators of targeted protein degradation and comprise a moiety that binds or interacts with cereblon and a moiety or substructure that binds or interacts with the target protein of interest, i.e., GSPT1 and/or IKZF1. The targeted degradation of the target protein of interest, i.e., GSPT1 and/or IKZF1, is believed to occur via a molecular glue mechanism as described herein above.

In one aspect, the present disclosure relates to compounds having a structure represented by a formula:

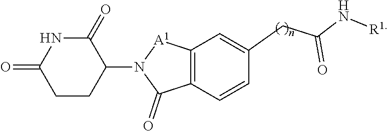

In a further aspect, the disclosure relates to compounds having a structure represented by a formula:

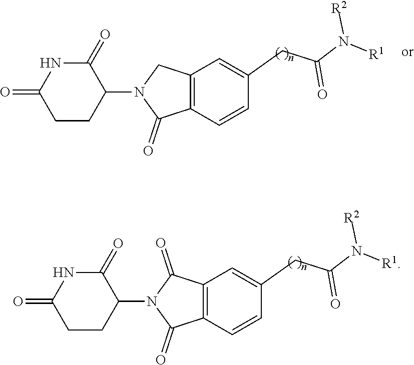

In a further aspect, the disclosure relates to compounds having a structure represented by a formula:

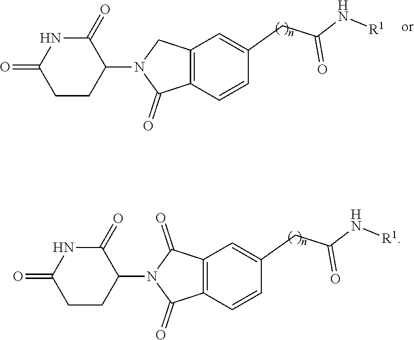

In a further aspect, the disclosure relates to compounds having a structure represented by a formula:

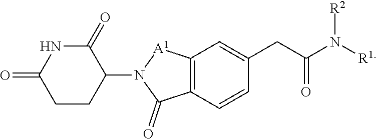

In a further aspect, the disclosure relates to compounds having a structure represented by a formula:

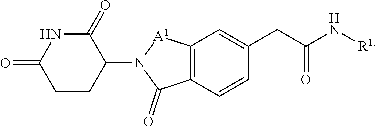

In a further aspect, the disclosure relates to compounds having a structure represented by a formula:

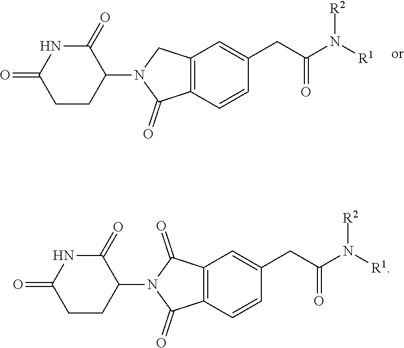

In a further aspect, the disclosure relates to compounds having a structure represented by a formula:

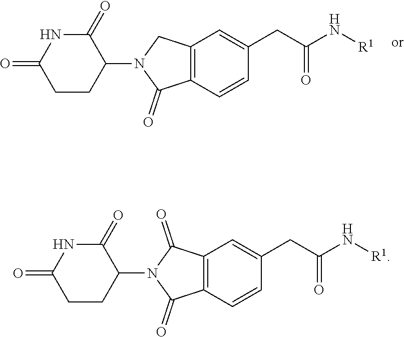

In a further aspect, the disclosure relates to compounds having a structure represented by a formula:

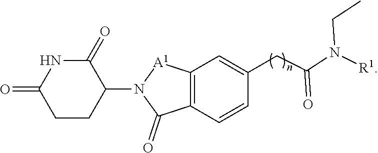

In a further aspect, the disclosure relates to compounds having a structure represented by a formula:

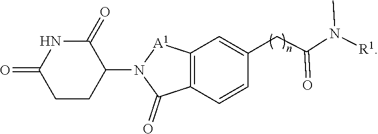

In a further aspect, the disclosure relates to compounds having a structure represented by a formula:

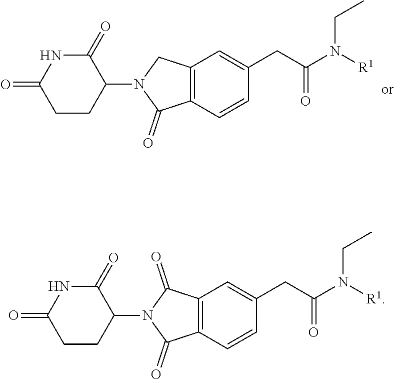

In a further aspect, the disclosure relates to compounds having a structure represented by a formula:

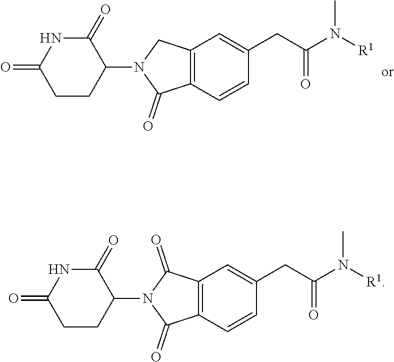

In a further aspect, the disclosure relates to compounds having a structure represented by a formula:

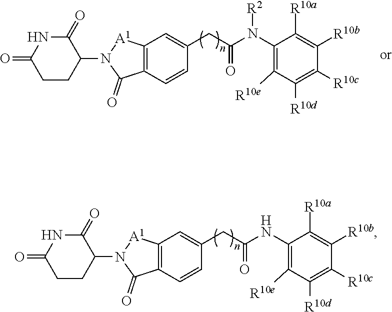

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

In a further aspect, the disclosure relates to compounds having a structure represented by a formula:

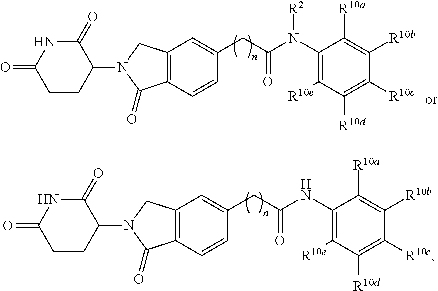

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

In a further aspect, the disclosure relates to compounds having a structure represented by a formula:

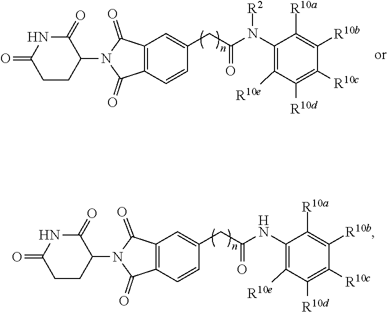

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

In a further aspect, the disclosure relates to compounds having a structure represented by a formula:

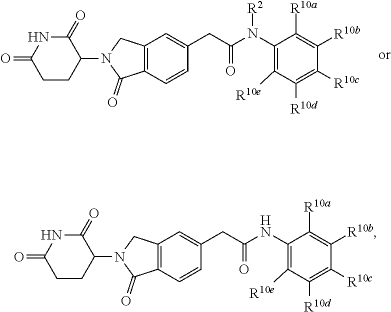

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

In a further aspect, the disclosure relates to compounds having a structure represented by a formula:

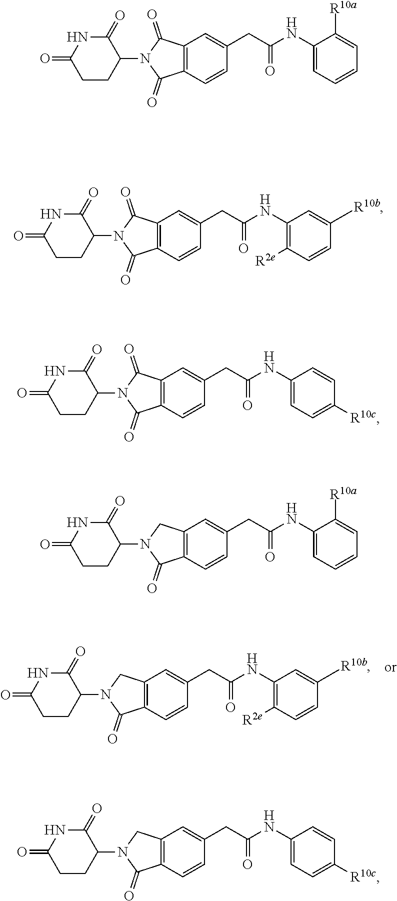

wherein each of R^{10a}, R^{10b}, R^{10c}, R^{10d} and R^{10e} is as defined herein.

In a further aspect, the disclosure relates to compounds having a structure represented by a formula:

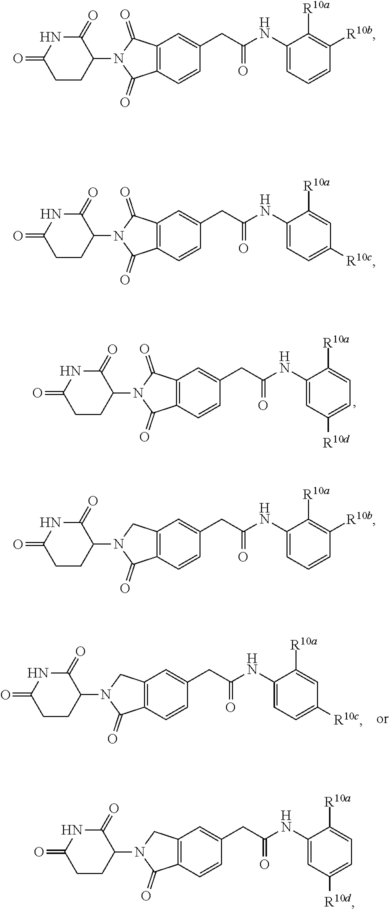

wherein each of R^{10a}, R^{10b}, R^{10c}, R^{10d} and R^{10e} is as defined herein.

In a further aspect, the disclosure relates to compounds having a structure represented by a formula:

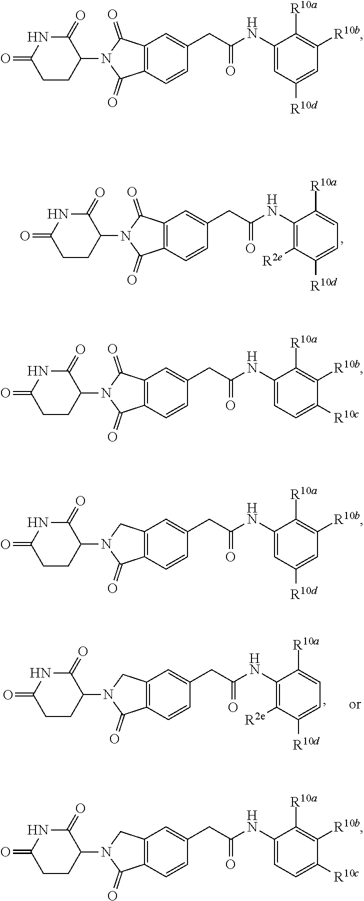

wherein each of R^{10a}, R^{10b}, R^{10c}, R^{10d}, and R^{10e} is as defined herein.

In a further aspect, a disclosed compound has a structure given by:

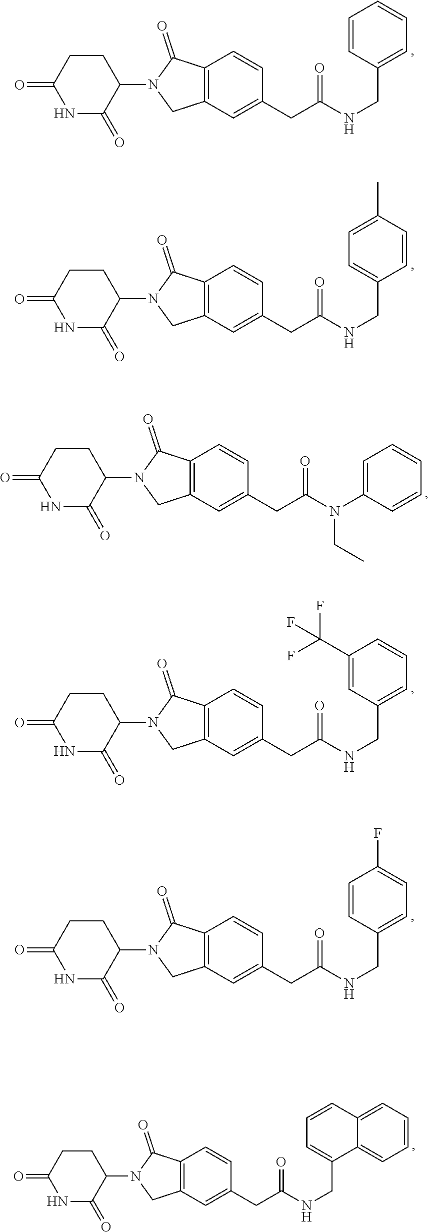 [SMILES: O=C(Cc1ccc2c(c1)CN(C1CCC(=O)NC1=O)C2=O)NCc1ccccc1 ; Cc1ccc(CNC(=O)Cc2ccc3c(c2)CN(C2CCC(=O)NC2=O)C3=O)cc1 ; CCN(C(=O)Cc1ccc2c(c1)CN(C1CCC(=O)NC1=O)C2=O)c1ccccc1 ; O=C(Cc1ccc2c(c1)CN(C1CCC(=O)NC1=O)C2=O)NCc1cccc(C(F)(F)F)c1 ; O=C(Cc1ccc2c(c1)CN(C1CCC(=O)NC1=O)C2=O)NCc1ccc(F)cc1 ; O=C(Cc1ccc2c(c1)CN(C1CCC(=O)NC1=O)C2=O)NCc1cccc2ccccc12]

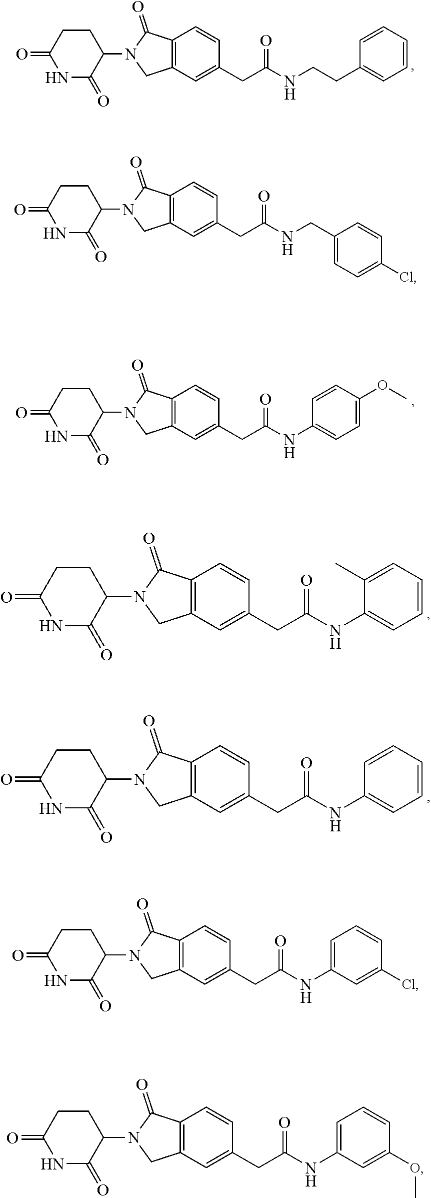

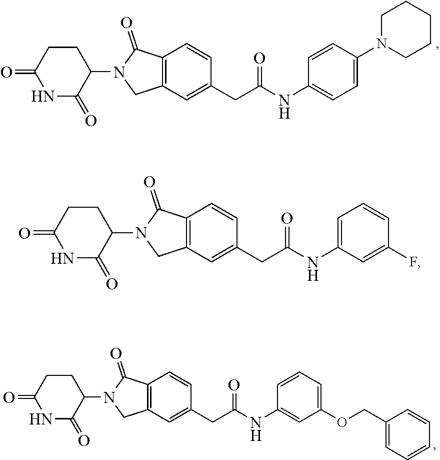 [SMILES: O=C1CCC(N2Cc3cc(CC(=O)Nc4ccc(N5CCCCC5)cc4)ccc3C2=O)C(=O)N1 ; O=C1CCC(N2Cc3cc(CC(=O)Nc4cccc(F)c4)ccc3C2=O)C(=O)N1 ; O=C1CCC(N2Cc3cc(CC(=O)Nc4cccc(OCc5ccccc5)c4)ccc3C2=O)C(=O)N1]

or a subgroup thereof.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further comprise their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³S, ¹⁸F and ³⁶Cl, respectively.

Compounds further comprise prodrugs thereof, and pharmaceutically acceptable salts of said compounds or of said prodrugs which contain the aforementioned isotopes and/or other isotopes of other atoms are within the scope of this disclosure. Certain isotopically-labelled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present disclosure and prodrugs thereof can generally be prepared by carrying out the procedures below, by substituting a readily available isotopically labelled reagent for a non-isotopically labelled reagent.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g., “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the disclosure to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### D. METHODS OF MAKING THE COMPOUNDS

In one aspect, the disclosure relates to methods of making compounds useful as antibacterial agents, which can be useful in the treatment of bacterial infections. In one aspect, the disclosure relates to the disclosed synthetic manipulations. In a further aspect, the disclosed compounds comprise the products of the synthetic methods described herein.

In a further aspect, the disclosed compounds comprise a compound produced by a synthetic method described herein. In a still further aspect, the disclosure comprises a pharmaceutical composition comprising a therapeutically effective amount of the product of the disclosed methods and a pharmaceutically acceptable carrier. In a still further aspect, the disclosure comprises a method for manufacturing a medicament comprising combining at least one product of the disclosed methods with a pharmaceutically acceptable carrier or diluent.

The compounds of this disclosure can be prepared by employing reactions as shown in the disclosed schemes, in addition to other standard manipulations that are known in the literature, exemplified in the experimental sections or clear to one skilled in the art. For clarity, examples having a fewer substituent can be shown where multiple substituents are allowed under the definitions disclosed herein. Thus, the following examples are provided so that the disclosure might be more fully understood, are illustrative only, and should not be construed as limiting.

It is contemplated that each disclosed method can further comprise additional steps, manipulations, and/or components. It is also contemplated that any one or more step, manipulation, and/or component can be optionally omitted from the disclosure. It is understood that a disclosed method can be used to provide the disclosed compounds. It is also understood that the products of the disclosed methods can be employed in the disclosed compositions, kits, and uses.

The general synthesis methods are as described herein below in the Examples. It is contemplated that each disclosed method can further comprise additional steps, manipulations, and/or components. It is also contemplated that any one or more step, manipulation, and/or component can be optionally omitted from the disclosure. It is understood that a disclosed method can be used to provide the disclosed compounds. It is also understood that the products of the disclosed methods can be employed in the disclosed methods of using.

### E. PHARMACEUTICAL COMPOSITIONS

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with an basoc reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C12H₂₄O₂ to C18H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g., 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g., about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present disclosure may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present disclosure include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present disclosure therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present disclosure include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the disclosure formulated in a pharmaceutically acceptable carrier may also be prepared, placed in an appropriate container, and labeled for treatment of an indicated condition.

Another patch system configuration which can be used by the present disclosure is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present disclosure is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require of modulation of cereblon protein an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

In the treatment conditions which require of modulation of GSPT1 activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

In the treatment conditions which require of modulation of IKZF1 activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

In the treatment conditions which require of inhibition of cellular proliferation an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating cereblon protein (e.g., treatment of one or more disorders associated with a cereblon function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for modulation of cereblon protein.

The present disclosure is further directed to a method for the manufacture of a medicament for modulating CRBN (e.g., treatment of one or more disorders associated with a cereblon function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for modulation of CRBN protein.

The present disclosure is further directed to a method for the manufacture of a medicament for inhibiting cellular proliferation (e.g., treatment of one or more disorders associated with a cereblon function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for inhibiting cellular proliferation.

The present disclosure is further directed to a method for the manufacture of a medicament for modulating cereblon protein (e.g., treatment of one or more disorders associated with a cereblon function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for modulation of cereblon protein.

The present disclosure is further directed to a method for the manufacture of a medicament for modulating CRBN (e.g., treatment of one or more disorders associated with a cereblon function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for modulation of CRBN protein.

The present disclosure is further directed to a method for the manufacture of a medicament for inhibiting cellular proliferation (e.g., treatment of one or more disorders associated with a cereblon function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for inhibiting cellular proliferation.

The present disclosure is further directed to a method for the manufacture of a medicament for modulating GSPT1 protein (e.g., treatment of one or more disorders associated with a GSPT1 function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for modulation of GSPT1 protein.

The present disclosure is further directed to a method for the manufacture of a medicament for modulating IKZF1 (e.g., treatment of one or more disorders associated with a IKZF1 function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for modulation of IKZF1 protein.

The present disclosure is further directed to a method for the manufacture of a medicament for inhibiting cellular proliferation (e.g., treatment of one or more disorders associated with a IKZF1 function or dysfunction, such as a cancer) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent, wherein the medicament is useful for inhibiting cellular proliferation.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and an additional therapeutic agent, e.g., an inhibitor of cellular proliferation or anti-cancer therapeutic. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### F. METHODS OF USING THE COMPOUNDS

In various aspects, the present disclosure provides methods of treatment comprising administration of a therapeutically effective amount of a disclosed compound or pharmaceutical composition as disclosed herein above to a subject in need thereof.

In a further aspect, the present disclosure provides methods for the treatment of a disorder of uncontrolled cellular proliferation in a mammal comprising the step of administering to the mammal a therapeutically effective amount of at least one disclosed compound or pharmaceutically acceptable salt thereof, or at least one disclosed pharmaceutical composition.

Also disclosed are methods for modulating of cereblon activity in a mammal comprising the step of administering to the mammal a therapeutically effective amount of at least one disclosed compound or pharmaceutically acceptable salt thereof, or at least one disclosed pharmaceutical composition.

Also disclosed are methods for modulating of GSPT1 activity in a mammal comprising the step of administering to the mammal a therapeutically effective amount of at least one disclosed compound or pharmaceutically acceptable salt thereof, or at least one disclosed pharmaceutical composition.

Also disclosed are methods for modulating of IKZF1 activity in a mammal comprising the step of administering to the mammal a therapeutically effective amount of at least one disclosed compound or pharmaceutically acceptable salt thereof, or at least one disclosed pharmaceutical composition.

Also disclosed are methods for modulating of cereblon activity in at least one cell, comprising the step of contacting the at least one cell with an effective amount of at least one disclosed compound, or a pharmaceutically acceptable salt thereof; or at least one disclosed pharmaceutical composition.

Also disclosed are methods for modulating of GSPT1 activity in at least one cell, comprising the step of contacting the at least one cell with an effective amount of at least one disclosed compound, or a pharmaceutically acceptable salt thereof; or at least one disclosed pharmaceutical composition.

Also disclosed are methods for modulating of IKZF1 activity in at least one cell, comprising the step of contacting the at least one cell with an effective amount of at least one disclosed compound, or a pharmaceutically acceptable salt thereof; or at least one disclosed pharmaceutical composition.

In a further aspect, the disorder of uncontrolled cellular proliferation is a cancer, e.g., a cancer is selected from a brain cancer, lung cancer, hematological cancer, bladder cancer, colon cancer, cervical cancer, ovarian cancer, squamous cell cancer, kidney cancer, peritoneal cancer, breast cancer, gastric cancer, colorectal cancer, prostate cancer, pancreatic cancer, genitourinary tract cancer, lymphatic system cancer, stomach cancer, larynx cancer, malignant melanoma, colorectal cancer, endometrial carcinoma, thyroid cancer, rhabdosarcoma, and combinations thereof. In a still further aspect, the cancer is a hematological cancer is selected from chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, Hodgkin lymphoma, non-Hodgkin lymphoma, and combinations thereof.

In a further aspect, the disclosed methods for the treatment of a disorder of uncontrolled cellular proliferation in a mammal further comprise the step of administering a therapeutically effective amount of at least one agent known to treat a cancer, e.g., uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In a further aspect, the disclosed methods for modulating of cereblon activity in at least one cell further comprise the step of contacting the at least one cell with an effective amount of at least one agent known to treat a cancer, e.g., uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In a further aspect, the disclosed methods for modulating of cereblon activity in a mammal comprising the step of administering to the mammal further comprise the step of the step of administering a therapeutically effective amount of at least one agent known to treat a cancer, e.g., uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In a further aspect, the disclosed methods for modulating of GSPT1 activity in at least one cell further comprise the step of contacting the at least one cell with an effective amount of at least one agent known to treat a cancer, e.g., uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In a further aspect, the disclosed methods for modulating of GSPT1 activity in a mammal comprising the step of administering to the mammal further comprise the step of the step of administering a therapeutically effective amount of at least one agent known to treat a cancer, e.g., uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In a further aspect, the disclosed methods for modulating of IKZF1 activity in at least one cell further comprise the step of contacting the at least one cell with an effective amount of at least one agent known to treat a cancer, e.g., uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In a further aspect, the disclosed methods for modulating of IKZF1 activity in a mammal comprising the step of administering to the mammal further comprise the step of the step of administering a therapeutically effective amount of at least one agent known to treat a cancer, e.g., uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

Also disclosed are uses of a disclosed compound, or a pharmaceutically acceptable salt thereof; a disclosed product of making, or a pharmaceutically acceptable salt thereof; or a disclosed pharmaceutical composition.

Also disclosed are uses of a disclosed compound, or a pharmaceutically acceptable salt thereof, in the manufacture of a medicament for the treatment of a disorder associated with a cereblon protein dysfunction in a mammal.

Also disclosed are uses of a disclosed compound, or a pharmaceutically acceptable salt thereof, in the manufacture of a medicament for the treatment of a disorder associated with a GSPT1 protein dysfunction in a mammal.

Also disclosed are uses of a disclosed compound, or a pharmaceutically acceptable salt thereof, in the manufacture of a medicament for the treatment of a disorder associated with a IKZF1 protein dysfunction in a mammal.

Also disclosed are uses of a disclosed compound, or a pharmaceutically acceptable salt thereof, in the manufacture of a medicament for the treatment of a disorder associated with a cellular proliferation dysfunction in a mammal, e.g., to inhibit cellular proliferation in a cancer cell, in a mammal comprising combining at least one disclosed compound, or a pharmaceutically acceptable salt thereof with a pharmaceutically acceptable carrier or diluent.

Also disclosed are methods for the manufacture of a medicament to modulate the cereblon protein in a mammal comprising combining at least one disclosed compound, or a pharmaceutically acceptable salt thereof with a pharmaceutically acceptable carrier or diluent.

Also disclosed are methods for the manufacture of a medicament to modulate the GSPT1 protein in a mammal comprising combining at least one disclosed compound, or a pharmaceutically acceptable salt thereof with a pharmaceutically acceptable carrier or diluent.

Also disclosed are methods for the manufacture of a medicament to modulate the IKZF1 protein in a mammal comprising combining at least one disclosed compound, or a pharmaceutically acceptable salt thereof with a pharmaceutically acceptable carrier or diluent.

Also disclosed are methods for the manufacture of a medicament to inhibit cellular proliferation, e.g., to inhibit cellular proliferation in a cancer cell, in a mammal comprising combining at least one disclosed compound, or a pharmaceutically acceptable salt thereof with a pharmaceutically acceptable carrier or diluent.

### G. KITS

In a further aspect, the present disclosure relates to kits comprising at least one disclosed compound, or a pharmaceutically acceptable salt thereof; or at least one disclosed pharmaceutical composition; and one or more of: (a) at least one agent known to increase cereblon activity; (b) at least one agent known to decrease cereblon activity; (c) at least one agent known to increase GSPT1 activity; (d) at least one agent known to decrease GSPT1 activity; (e) at least one agent known to increase IKZF1 activity; (f) at least one agent known to decrease IKZF1 activity; (g) at least one agent known to increase cellular proliferation; (h) at least one agent known to decrease cellular proliferation; (i) at least one agent known to treat a disorder associated with cereblon activity; (j) at least one agent known to treat a disorder associated with GSPT1 activity; (k) at least one agent known to treat a disorder associated with IKZF1 activity; (l) at least one agent known to treat a disorder of uncontrolled cellular proliferation; and/or (m) instructions for treating a disorder of uncontrolled cellular proliferation.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present disclosure also features such kits further containing instructions for use.

In a further aspect, the present disclosure also provides a pharmaceutical pack or kit comprising one or more containers filled with one or more of the ingredients of the pharmaceutical compositions of the disclosure. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### H. RESEARCH TOOLS

The disclosed compounds and pharmaceutical compositions have activity as modulators of cereblon protein. In a further aspect, the disclosed compounds and pharmaceutical compositions have activity as modulators of CRBN activity. In a still further aspect, the disclosed compounds and pharmaceutical compositions have activity as inhibitors of cellular proliferation. As such, the disclosed compounds are also useful as research tools.

Accordingly, one aspect of the present disclosure relates to a method of using a compound of the disclosure as a research tool, the method comprising conducting a biological assay using a compound of the disclosure. Compounds of the disclosure can also be used to evaluate new chemical compounds. Thus another aspect of the disclosure relates to a method of evaluating a test compound in a biological assay, comprising: (a) conducting a biological assay with a test compound to provide a first assay value; (b) conducting the biological assay with a compound of the disclosure to provide a second assay value; wherein step (a) is conducted either before, after or concurrently with step (b); and (c) comparing the first assay value from step (a) with the second assay value from step (b). Exemplary biological assays include a cereblon assay that can be conducted in vitro or in a cell culture system as disclosed herein, or alternatively, an assay of cellular proliferation using a cell-line and cellular proliferation assay as disclosed herein. Still another aspect of the disclosure relates to a method of studying a biological system, e.g., a model animal for a clinical condition, or biological sample comprising a cereblon protein the method comprising: (a) contacting the biological system or sample with a compound of the disclosure; and (b) determining the effects caused by the compound on the biological system or sample. A further aspect of the disclosure relates to a method of studying a biological system, e.g., a model animal for a clinical condition, or biological sample comprising a CRBN protein the method comprising: (a) contacting the biological system or sample with a compound of the disclosure; and (b) determining the effects caused by the compound on the biological system or sample. In various aspects, the disclosed compounds are useful as chemical probes for the study of CRBN in vitro and in vivo.

### I. REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
1. Chamberlain, P. P. and Hamann, L. G. Development of Targeted Protein Degradation Therapeutics. Nat Chem Biol 2019, 15 (10), 937-944.
2. Hanzl, A. and Winter, G. E. Targeted Protein Degradation: Current and Future Challenges. Current Opinion in Chemical Biology 2020, 56, 35-41.
3. Toure, M. and Crews, C. M. Small-Molecule PROTACS: New Approaches to Protein Degradation. Angew. Chem. Int. Ed. 2016, 55 (6), 1966-1973.
4. Schapira, M., et al. Targeted Protein Degradation: Expanding the Toolbox. Nat Rev Drug Discov 2019, 18 (12), 949-963.
5. Baek, K. and Schulman, B. A. Molecular Glue Concept Solidifies. Nat Chem Biol 2020, 16 (1), 2-3.
6. Fink, E. C. and Ebert, B. L. The Novel Mechanism of Lenalidomide Activity. Blood 2015, 126 (21), 2366-2369.
7. Kronke, J., et al. Lenalidomide Causes Selective Degradation of IKZF1 and IKZF3 in Multiple Myeloma Cells. Science 2014, 343 (6168), 301-305.
8. Han, T., et al. Anticancer Sulfonamides Target Splicing by Inducing RBM39 Degradation via Recruitment to DCAF15. Science 2017, 356 (6336), eaal3755.
9. Knott, M. M. L., et al. Targeting the Undruggable: Exploiting Neomorphic Features of Fusion Oncoproteins in Childhood Sarcomas for Innovative Therapies. Cancer Metastasis Rev 2019, 38 (4), 625-642.
10. Bushweller, J. H. Targeting Transcription Factors in Cancer—from Undruggable to Reality. Nat Rev Cancer 2019, 19 (11), 611-624.
11. Sievers, Q. L., et al. Defining the Human C2H2 Zinc Finger Degrome Targeted by Thalidomide Analogs through CRBN. Science 2018, 362 (6414), eaat0572.
12. Krönke, J., et al. Lenalidomide Induces Ubiquitination and Degradation of CK1α in Del(5q) MDS. Nature 2015, 523 (7559), 183-188.
13. Matyskiela, M. E., et al. A Cereblon Modulator (CC-220) with Improved Degradation of Ikaros and Aiolos. J. Med. Chem. 2018, 61 (2), 535-542.
14. Matyskiela, M. E., et al. A Novel Cereblon Modulator Recruits GSPT1 to the CRL4CRBN Ubiquitin Ligase. Nature 2016, 535 (7611), 252-257.
15. Sperling, A. S., et al. Patterns of Substrate Affinity, Competition, and Degradation Kinetics Underlie Biological Activity of Thalidomide Analogs. Blood 2019, 134 (2), 160-170.
16. Matyskiela, M. E., et al. Crystal Structure of the SALL4-Pomalidomide-Cereblon-DDB1 Complex. Nat Struct Mol Biol 2020, 27 (4), 319-322.
17. Yang, J., et al. Simple Structural Modifications Converting a Bona Fide MDM2 PROTAC Degrader into a Molecular Glue Molecule: A Cautionary Tale in the Design of PROTAC Degraders. J. Med. Chem. 2019, 62 (21), 9471-9487.
18. Ishoey, M., et al. Translation Termination Factor GSPT1 Is a Phenotypically Relevant Off-Target of Heterobifunctional Phthalimide Degraders. ACS Chem. Biol. 2018, 13 (3), 553-560.
19. Alexander, T. B., et al. The Genetic Basis and Cell of Origin of Mixed Phenotype Acute Leukaemia. Nature 2018, 562 (7727), 373-379.
20. Li, J.-F., et al. Transcriptional Landscape of B Cell Precursor Acute Lymphoblastic Leukemia Based on an International Study of 1,223 Cases. Proc Natl Acad Sci USA 2018, 115 (50), E11711-E11720.
21. Schwartz, J. R., et al. Comprehensive Genomic Profiling of Pediatric Therapy-Related Myeloid Neoplasms Identifies Mecom Dysregulation to Be Associated with Poor Outcome. Blood 2019, 134 (Supplement_1), 1394-1394.
22. Hinai, A. A. and Valk, P. J. M. Review: Aberrant EVI1 Expression in Acute Myeloid Leukaemia. Br J Haematol 2016, 172 (6), 870-878.
23. Northcott, P. A., et al. The Whole-Genome Landscape of Medulloblastoma Subtypes. Nature 2017, 547 (7663), 311-317.
24. Northcott, P. A., et al. Enhancer Hijacking Activates GFI1 Family Oncogenes in Medulloblastoma. Nature 2014, 511 (7510), 428-434.
25. Nowak, R. P., et al. Plasticity in Binding Confers Selectivity in Ligand-Induced Protein Degradation. Nat Chem Biol 2018, 14 (7), 706-714.
26. Leeson, P. D. and Springthorpe, B. The Influence of Drug-like Concepts on Decision-Making in Medicinal Chemistry. Nat Rev Drug Discov 2007, 6 (11), 881-890.
27. Kronke, J., et al. Lenalidomide Causes Selective Degradation of IKZF1 and IKZF3 in Multiple Myeloma Cells. Science 2014, 343 (6168), 301-305.
28. Lu, G., et al. Elucidating the Mechanism of Action of CC-90009, a Novel Cereblon E3 Ligase Modulator, in AML Via Genome-Wide CRISPR Screen. Blood 2019, 134 (Supplement_1), 405-405. https://doi.org/10.1182/blood-2019-125492.
29. Lu, G.; Lopez-Girona, A.; and Filvaroff, E. Methods for Treating Cancer and the Use of Biomarkers as a Predictor of Clinical Sensitivity to Therapies. U.S. Pat. Publ. No. 2017/199193A1 (published Jul. 13, 2017).
30. Bunnage, M. E.; Chekler, E. L. P.; and Jones, L. H. Target Validation Using Chemical Probes. Nat Chem Biol 2013, 9 (4), 195-199.
31. Chauvin, C.; Salhi, S.; and Jean-Jean, O. Human Eukaryotic Release Factor 3a Depletion Causes Cell Cycle Arrest at G1 Phase through Inhibition of the MTOR Pathway. MCB 2007, 27 (16), 5619-5629.
32. Uy, G. L., et al. Clinical Activity of CC-90009, a Cereblon E3 Ligase Modulator and First-in-Class GSPT1 Degrader, As a Single Agent in Patients with Relapsed or Refractory Acute Myeloid Leukemia (R/R AML): First Results from a Phase I Dose-Finding Study. Blood 2019, 134 (Supplement_1), 232-232.
33. Hopkins, A. L.; Groom, C. R.; and Alex, A. Ligand Efficiency: A Useful Metric for Lead Selection. Drug Discovery Today 2004, 9 (10), 430-431.
34. Abadzapatero, C. and Metz, J. Ligand Efficiency Indices as Guideposts for Drug Discovery. Drug Discovery Today 2005, 10 (7), 464-469.
35. Bai, B., et al. Deep Multilayer Brain Proteomics Identifies Molecular Networks in Alzheimer's Disease Progression. Neuron 2020, 106 (4), 700. https://doi.org/10.1016/j.neuron.2020.04.031.
36. Connelly, J. P. and Pruett-Miller, S. M. CRIS.Py: A Versatile and High-Throughput Analysis Program for CRISPR-Based Genome Editing. Sci Rep 2019, 9 (1), 4194.
37. Bai, B., et al. Deep Profiling of Proteome and Phosphoproteome by Isobaric Labeling, Extensive Liquid Chromatography, and Mass Spectrometry. In Methods in Enzymology; Elsevier, 2017; Vol. 585, pp 377-395. https://doi.org/10.1016/bs.mie.2016.10.007.
38. Larson, B.; Banks, P.; and Sherman, H.; Rothenberg, M. Automation of Cell-Based Drug Absorption Assays in 96-Well Format Using Permeable Support Systems. J Lab Autom. 2012, 17 (3), 222-232.
39. Uchida, M., et al. A Modified Fast (4 Day) 96-Well Plate Caco-2 Permeability Assay. Journal of Pharmacological and Toxicological Methods 2009, 59 (1), 39-43.
40. Di, L., et al. High Throughput Microsomal Stability Assay for Insoluble Compounds. International Journal of Pharmaceutics 2006, 317 (1), 54-60.
41. Di, L., et al. Applications of High Throughput Microsomal Stability Assay in Drug Discovery. CCHTS 2008, 11 (6), 469-476. https://doi.org/10.2174/138620708784911429.

### J. ASPECTS

The following listing of exemplary aspects supports and is supported by the disclosure provided herein.

Aspect 1. A compound having a structure represented by a formula:

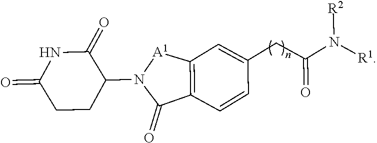

wherein each of A¹ is selected from —(C═O)— and —(CH₂)—; wherein n is selected from 1, 2, and 3; wherein R¹ is selected from —(C1-C3 alkanediyl)-phenyl and —(C1-C3 alkanediyl)-naphthyl; and wherein R¹ is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1—C3 haloalkyl), C3-C8 cycloalkyl, C1-C6 alkyl, -A²⁰-A¹⁰, and -A¹⁰; wherein -A¹⁰ is selected from substituted naphthyl, substituted phenyl, substituted cycloalkyl, and substituted heterocycloalkyl; and wherein -A²⁰- is selected from —(CH₂)ₓ—, —(O—CH₂)ₓ—, —(NH—CH₂)ₓ—, —(CH₂—O)ₓ—, and —(CH₂—NH)ₓ—; wherein x is selected from 0, 1, 2, and 3; and wherein R² is selected from hydrogen, substituted C1-C10 alkyl, and substituted C1-C10 heteroalkyl; or a pharmaceutically acceptable salt thereof.

Aspect 2. The compound of Aspect 1, wherein A¹ is —(C═O)—.

Aspect 3. The compound of Aspect 1, wherein A¹ is —(CH₂)—.

Aspect 4. The compound of any one of Aspect 1-Aspect 3, wherein n is selected from 1 and 2.

Aspect 5. The compound of Aspect 4, wherein n is 1.

Aspect 6. The compound of Aspect 4, wherein n is 2.

Aspect 7. The compound of any one of Aspect 1-Aspect 3, wherein n is selected from 2 and 3.

Aspect 8. The compound of any one of Aspect 1-Aspect 7, wherein R¹ is —(CH₂) m-phenyl; and wherein the phenyl is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰; and wherein m is selected from 0, 1, 2, and 3.

Aspect 9. The compound of Aspect 8, wherein m is 0.

Aspect 10. The compound of Aspect 8, wherein m is selected from 0, 1, and 2.

Aspect 11. The compound of Aspect 8, wherein m is selected from 1 and 2.

Aspect 12. The compound of Aspect 8, wherein m is selected from 0 and 1.

Aspect 13. The compound of Aspect 8, wherein R¹ is -phenyl; and wherein the phenyl is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 14. The compound of Aspect 8, wherein R¹ is —(CH₂)-phenyl; and wherein the phenyl is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 15. The compound of Aspect 8, wherein R¹ is —(CH₂)₂-phenyl; and wherein the phenyl is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 16. The compound of Aspect 8, wherein R¹ is —(CH₂)₃-phenyl; and wherein the phenyl is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 17. The compound of any one of Aspect 8-Aspect 16, wherein the phenyl is unsubstituted.

Aspect 18. The compound of any one of Aspect 8-Aspect 16, wherein the phenyl is substituted with one or more group selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 19. The compound of Aspect 18, wherein the phenyl has a structure represented by a formula:

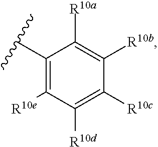

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 20. The compound of Aspect 19, wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 21. The compound of Aspect 20, wherein each of R^{10a}, R^{10b}, R^{10c}, R^{10d} and R^{10e} is independently selected from hydrogen, —F, —Cl, —Br, —CH₂F, —CHF₂, —CF₃, —CH₂Cl, —CHCl₂, —CCl₃, —CH₂Br, —CHBr₂, —CBr₃, —OCH₂F, —OCHF₂, —OCF₃, —OCH₂Cl, —OCHCl₂, —OCCl₃, —OCH₂Br, —OCHBr₂, —OCBr₃, —OCH₃, —OCH₂CH₃, —CH₂OH, —(CH₂)₂OH, —NHCH₃, —NHCH₂CH₃, —N(CH₃)₂, methyl, ethyl, propyl, isopropyl, butyl, isobutyl, and tert-butyl.

Aspect 22. The compound of Aspect 20, wherein each of R^{10a}, R^{10b}, R^{10c}, R^{10d} and R^{10e} is independently selected from hydrogen, —F, —Cl, —CCl₃, —OCF₃, —OCCl₃, —OCH₃, methyl, ethyl, propyl, isopropyl, butyl, isobutyl, and tert-butyl.

Aspect 23. The compound of Aspect 20, wherein each of R^{10a}, R^{10b}, R^{10c}, R^{10d} and R^{10e} is independently selected from hydrogen, —F, —Cl, —OCF₃, methyl, ethyl, and tert-butyl.

Aspect 24. The compound of Aspect 20, wherein each of R^{10a}, R^{10b}, R^{10c}, R^{10d} and R^{10e} is independently selected from hydrogen, —F, —Cl, —OCF₃, and methyl.

Aspect 25. The compound of Aspect 19, wherein four of R^{10a}, R^{10b}, R^{10c}, R^{10d} and R^{10e} are hydrogen.

Aspect 26. The compound of Aspect 19, wherein three of R^{10a}, R^{10b}, R^{10c}, R^{10d} and R^{10e} are hydrogen.

Aspect 27. The compound of Aspect 19, wherein two of R^{10a}, R^{10b}, R^{10c}, R^{10d} and R^{10e} are hydrogen.

Aspect 28. The compound of Aspect 19, wherein at least one of R^{10a}, R^{10b}, R^{10c}, R^{10d} and R^{10e} is independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 29. The compound of Aspect 28, wherein one of R^{10a}, R^{10b}, R¹, R^{10d} and R^{10e} is independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C6 cycloalkyl, C1-C3 alkyl, -A²⁰-A¹⁰, and -A¹⁰.

Aspect 30. The compound of Aspect 28, wherein one of R^{10a}, R^{10b}, R¹, R^{10d} and R^{10e} is independently selected from —F, —Cl, —Br, —SF₅, —CN, —N₃, —CN, —CH₂F, —CHF₂, —CF₃, —CH₂C1, —CHCl₂, —CCl₃, —CH₂Br, —CHBr₂, —CBr₃, —CH₂CH₂F, —CH₂CHF₂, —CH₂CF₃, —CH₂CH₂C1, —CH₂CHCl₂, —CH₂CCl₃, —CH₂CH₂Br, —CH₂CHBr₂, —CH₂CBr₃, —OCH₂F, —OCHF₂, —OCF₃, —OCH₂C1, —OCHCl₂, —OCCl₃, —OCH₂Br, —OCHBR¹⁰, —OCBr₃, —OCH₂CH₂F, —OCH₂CHF₂, —OCH₂CF₃, —OCH₂CH₂C1, —OCH₂CHCl₂, —OCH₂CCl₃, —OCH₂CH₂Br, —OCH₂CHBR¹⁰, —OCH₂CBr₃, —OCH₃, —OCH₂CH₃, —CH₂OH, —(CH₂)₂OH, —NHCH₃, —NHCH₂CH₃, —N(CH₃)₂, methyl, ethyl, propyl, isopropyl, butyl, isobutyl, and tert-butyl.

Aspect 31. The compound of Aspect 28, wherein one of R^{10a}, R^{10b}, R^{10c}, R^{10d} and R^{10e} is independently selected from —F, —Cl, —Br, —CH₂F, —CHF₂, —CF₃, —CH₂Cl, —CHCl₂, —CCl₃, —CH₂Br, —CHBr₂, —CBr₃, —OCH₂F, —OCHF₂, —OCF₃, —OCH₂Cl, —OCHCl₂, —OCCl₃, —OCH₂Br, —OCHBr₂, —OCBr₃, —OCH₃, —OCH₂CH₃, —CH₂OH, —(CH₂)₂OH, —NHCH₃, —NHCH₂CH₃, —N(CH₃)₂, methyl, ethyl, propyl, isopropyl, butyl, isobutyl, and tert-butyl.

Aspect 32. The compound of Aspect 28, wherein one of R^{10a}, R^{10b}, R^{10c}, R^{10d} and R^{10e} is independently selected from —F, —Cl, —CCl₃, —OCF₃, —OCCl₃, —OCH₃, methyl, ethyl, propyl, isopropyl, butyl, isobutyl, and tert-butyl.

Aspect 33. The compound of Aspect 28, wherein one of R^{10a}, R^{10b}, R^{10c}, R^{10d} and R^{10e} is independently selected from —F, —Cl, —OCF₃, methyl, ethyl, and tert-butyl.

Aspect 34. The compound of Aspect 28, wherein one of R^{10a}, R^{10b}, R^{10c}, R^{10d} and R^{10e} is independently selected from —F, —Cl, —OCF₃, and methyl.

Aspect 35. The compound of Aspect 19, wherein two of R^{10a}, R^{10b}, R^{10c}, R^{10d} and R^{10e} are independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 36. The compound of Aspect 35, wherein one or two of R^{10a}, R^{10b}, R^{10c}, R^{10d} and R^{10e} is independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C6 cycloalkyl, C1-C3 alkyl, -A²⁰-A¹⁰, and -A¹⁰.

Aspect 37. The compound of Aspect 35, wherein one of R^{10a}, R^{10b}, R^{10c}, R^{10d} and R^{10e} is independently selected from —F, —Cl, —Br, —SF₅, —CN, —N₃, —CN, —CH₂F, —CHF₂, —CF₃, —CH₂C1, —CHCl₂, —CCl₃, —CH₂Br, —CHBr₂, —CBr₃, —CH₂CH₂F, —CH₂CHF₂, —CH₂CF₃, —CH₂CH₂C1, —CH₂CHCl₂, —CH₂CCl₃, —CH₂CH₂Br, —CH₂CHBr₂, —CH₂CBr₃, —OCH₂F, —OCHF₂, —OCF₃, —OCH₂Cl, —OCHCl₂, —OCCl₃, —OCH₂Br, —OCHBR¹⁰, —OCBr₃, —OCH₂CH₂F, —OCH₂CHF₂, —OCH₂CF₃, —OCH₂CH₂C1, —OCH₂CHCl₂, —OCH₂CCl₃, —OCH₂CH₂Br, —OCH₂CHBR¹⁰, —OCH₂CBr₃, —OCH₃, —OCH₂CH₃, —CH₂OH, —(CH₂)₂OH, —NHCH₃, —NHCH₂CH₃, —N(CH₃)₂, methyl, ethyl, propyl, isopropyl, butyl, isobutyl, and tert-butyl.

Aspect 38. The compound of Aspect 35, wherein one of R^{10a}, R^{10b}, R^{10c}, R^{10d} and R^{10e} is independently selected from —F, —Cl, —Br, —CH₂F, —CHF₂, —CF₃, —CH₂C1, —CHCl₂, —CCl₃, —CH₂Br, —CHBr₂, —CBr₃, —OCH₂F, —OCHF₂, —OCF₃, —OCH₂C1, —OCHCl₂, —OCCl₃, —OCH₂Br, —OCHBr₂, —OCBr₃, —OCH₃, —OCH₂CH₃, —CH₂OH, —(CH₂)₂OH, —NHCH₃, —NHCH₂CH₃, —N(CH₃)₂, methyl, ethyl, propyl, isopropyl, butyl, isobutyl, and tert-butyl.

Aspect 39. The compound of Aspect 35, wherein one of R^{10a}, R^{10b}, R^{10c}, R^{10d} and R^{10e} is independently selected from —F, —Cl, —CCl₃, —OCF₃, —OCCl₃, —OCH₃, methyl, ethyl, propyl, isopropyl, butyl, isobutyl, and tert-butyl.

Aspect 40. The compound of Aspect 35, wherein one of R^{10a}, R^{10b}, R^{10c}, R^{10d} and R^{10e} is independently selected from —F, —Cl, —OCF₃, methyl, ethyl, and tert-butyl.

Aspect 41. The compound of Aspect 35, wherein one of R^{10a}, R^{10b}, R^{10c}, R^{10d} and R^{10e} is independently selected from —F, —Cl, —OCF₃, and methyl.

Aspect 42. The compound of Aspect 19, wherein three of R^{10a}, R^{10b}, R^{10c}, R^{10d} and R^{10e} are independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, and phenyl.

Aspect 43. The compound of Aspect 42, wherein one, two, or three of R^{10a}, R^{10b}, R^{10c}, R^{10d} and R^{10e} is independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C6 cycloalkyl, C1-C3 alkyl, -A²⁰-A¹⁰, and -A¹⁰.

Aspect 44. The compound of Aspect 42, wherein one of R^{10a}, R^{10b}, R^{10c}, R^{10d} and R^{10e} is independently selected from —F, —Cl, —Br, —SF₅, —CN, —N₃, —CN, —CH₂F, —CHF₂, —CF₃, —CH₂C1, —CHCl₂, —CCl₃, —CH₂Br, —CHBr₂, —CBr₃, —CH₂CH₂F, —CH₂CHF₂, —CH₂CF₃, —CH₂CH₂C1, —CH₂CHCl₂, —CH₂CCl₃, —CH₂CH₂Br, —CH₂CHBr₂, —CH₂CBr₃, —OCH₂F, —OCHF₂, —OCF₃, —OCH₂C1, —OCHCl₂, —OCCl₃, —OCH₂Br, —OCHBR¹⁰, —OCBr₃, —OCH₂CH₂F, —OCH₂CHF₂, —OCH₂CF₃, —OCH₂CH₂C1, —OCH₂CHCl₂, —OCH₂CCl₃, —OCH₂CH₂Br, —OCH₂CHBR¹⁰, —OCH₂CBr₃, —OCH₃, —OCH₂CH₃, —CH₂OH, —(CH₂)₂OH, —NHCH₃, —NHCH₂CH₃, —N(CH₃)₂, methyl, ethyl, propyl, isopropyl, butyl, isobutyl, and tert-butyl.

Aspect 45. The compound of Aspect 42, wherein one of R^{10a}, R^{10b}, R^{10c}, R^{10d} and R^{10e} is independently selected from —F, —C, —Br, —CH₂F, —CHF₂, —CF₃, —CH₂Cl, —CHCl₂, —CCl₃, —CH₂Br, —CHBr₂, —CBr₃, —OCH₂F, —OCHF₂, —OCF₃, —OCH₂C1, —OCHCl₂, —OCCl₃, —OCH₂Br, —OCHBr₂, —OCBr₃, —OCH₃, —OCH₂CH₃, —CH₂OH, —(CH₂)₂OH, —NHCH₃, —NHCH₂CH₃, —N(CH₃)₂, methyl, ethyl, propyl, isopropyl, butyl, isobutyl, and tert-butyl.

Aspect 46. The compound of Aspect 42, wherein one of R^{10a}, R^{10b}, R^{10c}, R^{10d} and R^{10e} is independently selected from —F, —Cl, —CCl₃, —OCF₃, —OCCl₃, —OCH₃, methyl, ethyl, propyl, isopropyl, butyl, isobutyl, and tert-butyl.

Aspect 47. The compound of Aspect 42, wherein one of R^{10a}, R^{10b}, R^{10c}, R^{10d} and R^{10e} is independently selected from —F, —Cl, —OCF₃, methyl, ethyl, and tert-butyl.

Aspect 48. The compound of Aspect 42, wherein one of R^{10a}, R^{10b}, R^{10c}, R^{10d} and R^{10e} is independently selected from —F, —Cl, —OCF₃, and methyl.

Aspect 49. The compound of any one of Aspect 1-Aspect 48, wherein -A¹⁰ is selected from unsubstituted naphthyl, unsubstituted phenyl, unsubstituted cycloalkyl, and unsubstituted heterocycloalkyl.

Aspect 50. The compound of Aspect 49, wherein the unsubstituted cycloalkyl is selected from cyclopropyl, cyclobutyl, cyclopentyl, cyclohexyl, and cycloheptyl.

Aspect 51. The compound of Aspect 50, wherein the unsubstituted cycloalkyl is selected from cyclopropyl, cyclobutyl, cyclopentyl, and cyclohexyl.

Aspect 52. The compound of Aspect 51, wherein the unsubstituted cycloalkyl is selected from cyclopropyl, cyclobutyl, and cyclopentyl.

Aspect 53. The compound of Aspect 50, wherein the unsubstituted cycloalkyl is cycloheptyl.

Aspect 54. The compound of Aspect 50, wherein the unsubstituted cycloalkyl is cyclohexyl.

Aspect 55. The compound of Aspect 50, wherein the unsubstituted cycloalkyl is cyclopentyl.

Aspect 56. The compound of Aspect 50, wherein the unsubstituted cycloalkyl is cyclobutyl.

Aspect 57. The compound of Aspect 50, wherein the unsubstituted cycloalkyl is cyclopropyl.

Aspect 58. The compound of Aspect 49, wherein the unsubstituted heterocycloalkyl is selected from pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

Aspect 59. The compound of Aspect 58, wherein the unsubstituted heterocycloalkyl is piperidinyl.

Aspect 60. The compound of Aspect 49, wherein -A¹⁰ is selected from unsubstituted naphthyl, and unsubstituted phenyl.

Aspect 61. The compound of Aspect 60, wherein -A¹⁰ is unsubstituted phenyl.

Aspect 62. The compound of any one of Aspect 1-Aspect 61, x is selected from 0, 1, and 2.

Aspect 63. The compound of Aspect 62, wherein x is selected from 0 and 1.

Aspect 64. The compound of Aspect 62, wherein x is selected from 1 and 2.

Aspect 65. The compound of any one of Aspect 1-Aspect 61, x is selected from 1, 2, and 3.

Aspect 66. The compound of Aspect 65, wherein x is selected from 1 and 2.

Aspect 67. The compound of Aspect 65, wherein x is selected from 2 and 3.

Aspect 68. The compound of any one of Aspect 1-Aspect 67, wherein -A²⁰ is selected from —(CH₂)ₓ—, —(O—CH₂)ₓ—, and —(NH—CH₂)ₓ—.

Aspect 69. The compound of Aspect 68, wherein -A²⁰ is —(CH₂)ₓ—.

Aspect 70. The compound of Aspect 68, wherein -A²⁰ is —(O—CH₂)ₓ—.

Aspect 71. The compound of Aspect 68, wherein -A²⁰ is —(NH—CH₂)ₓ—.

Aspect 72. The compound of any one of Aspect 1-Aspect 71, wherein R² is selected from hydrogen, methyl, and ethyl.

Aspect 73. The compound of Aspect 72, wherein R² is hydrogen.

Aspect 74. The compound of Aspect 72, wherein R² is methyl.

Aspect 75. The compound of Aspect 72, wherein R² is ethyl.

Aspect 76. The compound of Aspect 1, having a structure represented by a formula:

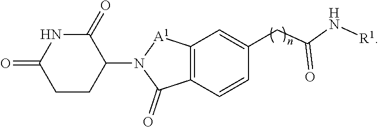

Aspect 77. The compound of Aspect 1, having a structure represented by a formula:

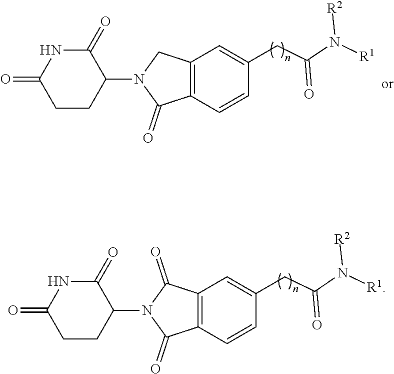

Aspect 78. The compound of Aspect 1, having a structure represented by a formula:

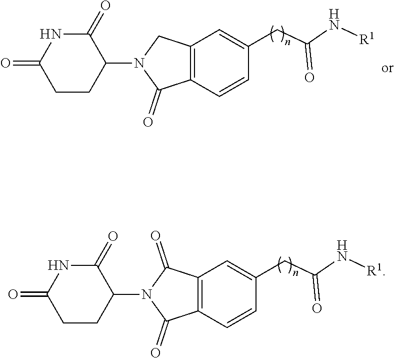

Aspect 79. The compound of Aspect 1, having a structure represented by a formula:

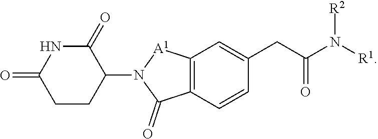

Aspect 80. The compound of Aspect 1, having a structure represented by a formula:

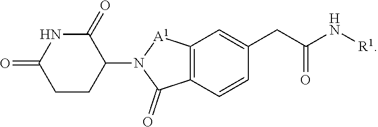

Aspect 81. The compound of Aspect 1, having a structure represented by a formula:

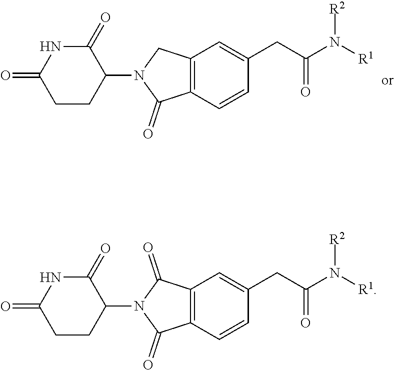

Aspect 82. The compound of Aspect 1, having a structure represented by a formula:

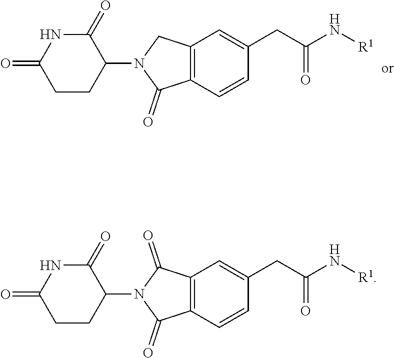

Aspect 83. The compound of Aspect 1, having a structure represented by a formula:

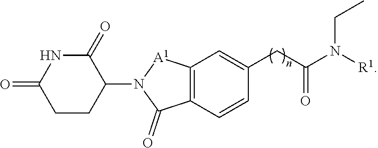

Aspect 84. The compound of Aspect 1, having a structure represented by a formula:

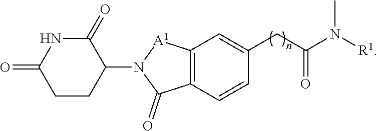

Aspect 85. The compound of Aspect 1, having a structure represented by a formula:

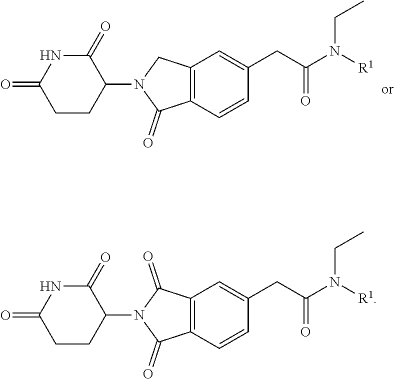

Aspect 86. The compound of Aspect 1, having a structure represented by a formula:

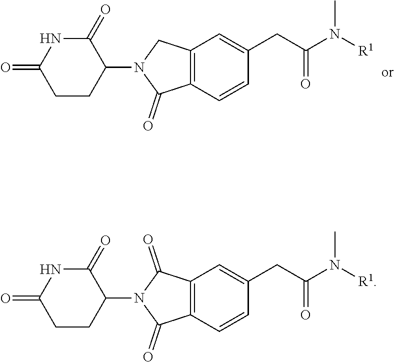

Aspect 87. The compound of Aspect 1, having a structure represented by a formula:

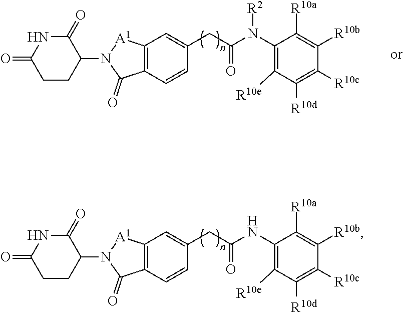

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 88. The compound of Aspect 1, having a structure represented by a formula:

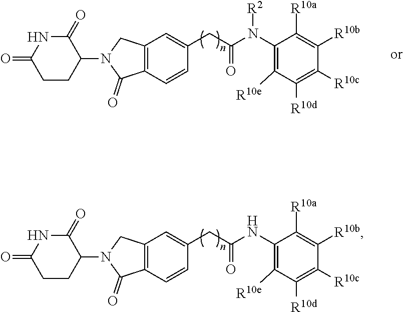

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 89. The compound of Aspect 1, having a structure represented by a formula:

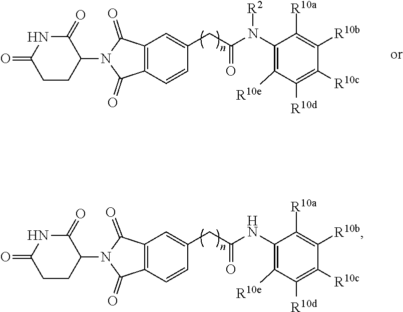

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 90. The compound of Aspect 1, having a structure represented by a formula:

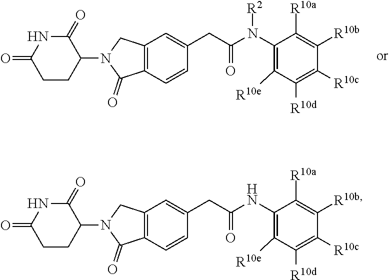

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 91. The compound of Aspect 1, having a structure represented by a formula:

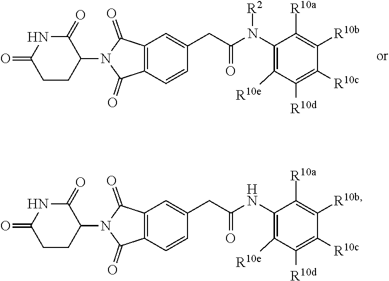

wherein each of R^{10a}, R^{10b}, R^{10c}, R^{10d} and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

Aspect 92. The compound of Aspect 87, having a structure represented by a formula:

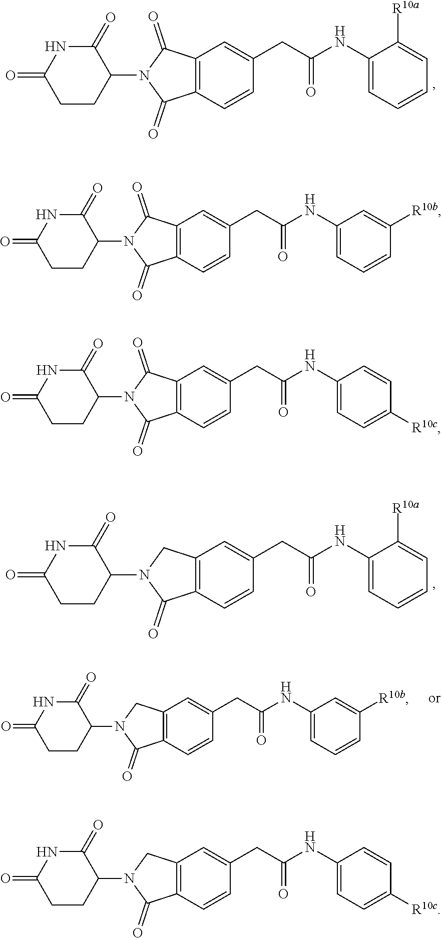

Aspect 93. The compound of Aspect 87, having a structure represented by a formula:

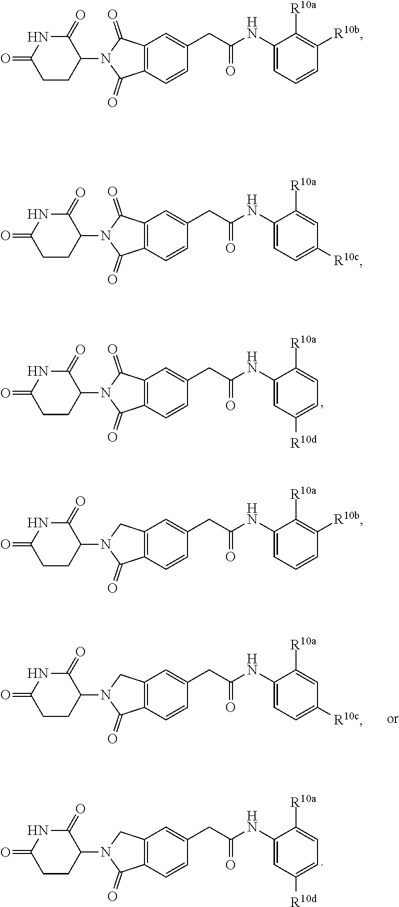

Aspect 94. The compound of Aspect 87, having a structure represented by a formula:

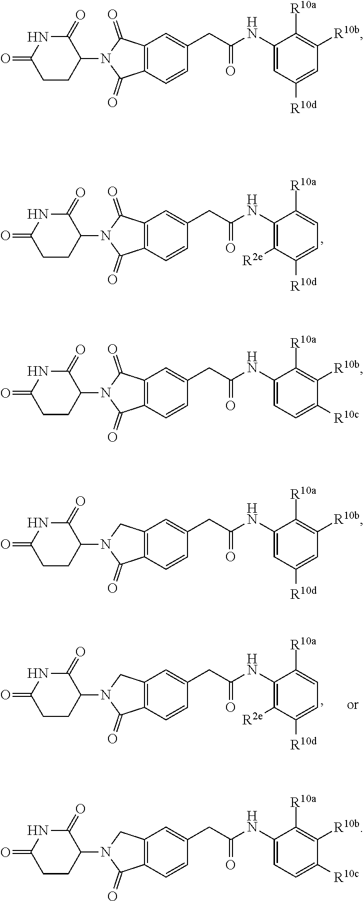

Aspect 95. The compound of Aspect 1, having a structure represented by a formula:

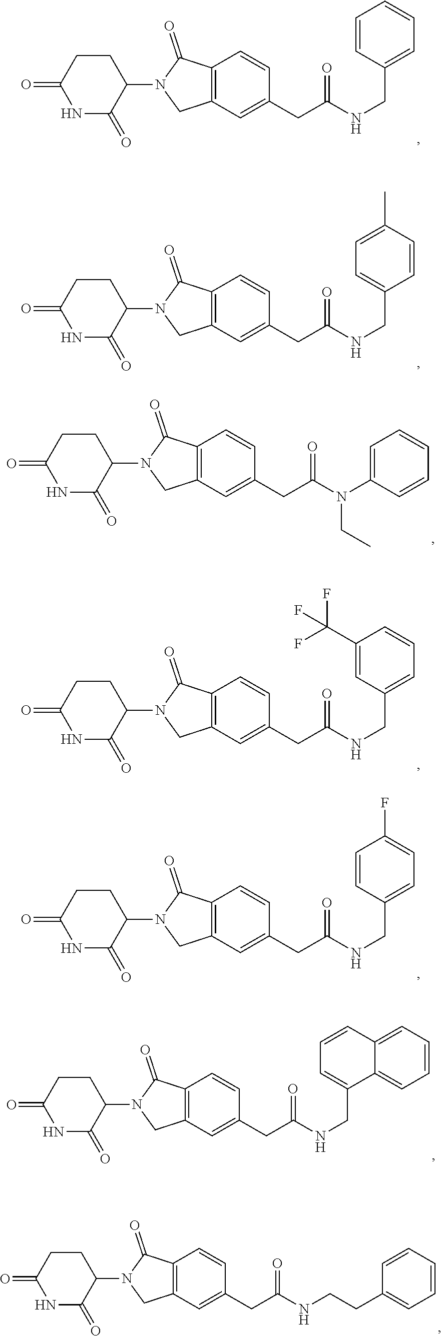

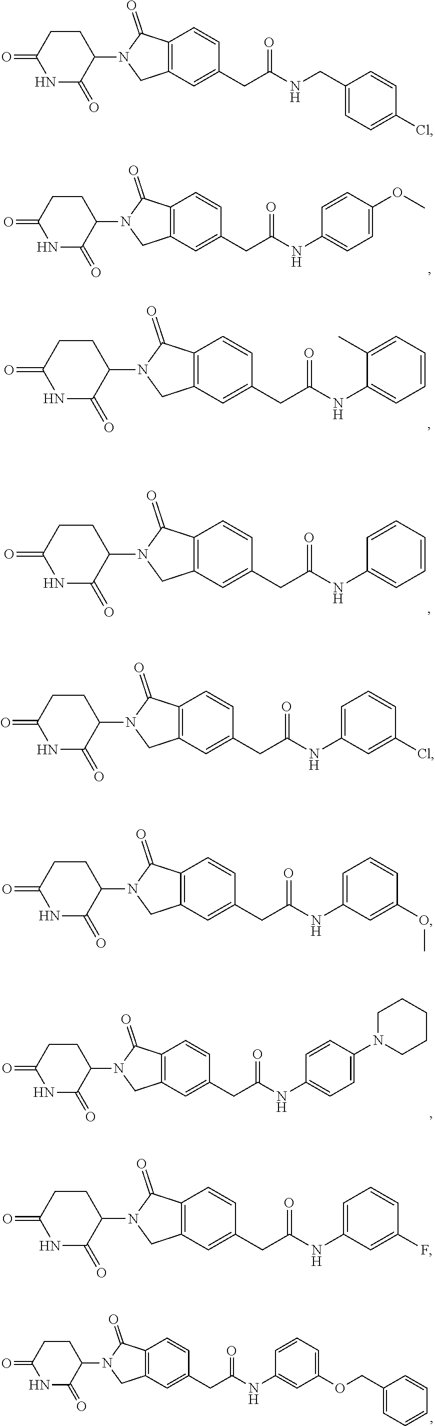

or a subgroup thereof.

Aspect 96. A pharmaceutical composition comprising a therapeutically effective amount of a compound of any one of Aspect 1-Aspect 95, or a pharmaceutically acceptable salt, solvate, or polymorph thereof, and a pharmaceutically acceptable carrier.

Aspect 97. The pharmaceutical composition of Aspect 96, further comprising at least one agent known to treat a cancer.

Aspect 98. The pharmaceutical composition of Aspect 97, wherein the at least one agent known to treat a cancer is a hormone therapy agent; an alkylating agent, an antimetabolite agent, an antineoplastic antibiotic agent, a mitotic inhibitor agent, a mTor inhibitor agent, other chemotherapeutic agent, or combinations thereof.

Aspect 99. The pharmaceutical composition of Aspect 98, wherein the at least one agent known to treat a cancer is a hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt thereof.

Aspect 100. The pharmaceutical composition of Aspect 98, wherein the at least one agent known to treat a cancer is a antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

Aspect 101. The pharmaceutical composition of Aspect 98, wherein the at least one agent known to treat a cancer is an antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

Aspect 102. The pharmaceutical composition of Aspect 98, wherein the at least one agent known to treat a cancer is an alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt.

Aspect 103. The pharmaceutical composition of Aspect 98, wherein the at least one agent known to treat a cancer is a mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt.

Aspect 104. The pharmaceutical composition of Aspect 98, wherein the at least one agent known to treat a cancer is a mTor inhibitor agent is selected from one or more of the group consisting of everolimus, siroliumus, and temsirolimus, or a pharmaceutically acceptable salt thereof.

Aspect 105. The pharmaceutical composition of Aspect 98, wherein the at least one agent known to treat a cancer is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17?-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

Aspect 106. A method for the treatment of a disorder of uncontrolled cellular proliferation in a mammal comprising the step of administering to the mammal a therapeutically effective amount of at least one compound of any one of Aspect 1-Aspect 95, or a pharmaceutically acceptable salt thereof; or the pharmaceutical composition of any one of Aspect 96-Aspect 105.

Aspect 107. The method of Aspect 106, wherein the mammal is a human.

Aspect 108. The method of Aspect 106, wherein the mammal has been diagnosed with a need for treatment of the disorder of uncontrolled cellular proliferation prior to the administering step.

Aspect 109. The method of Aspect 106, further comprising the step of identifying a mammal in need of treatment of the disorder of uncontrolled cellular proliferation.

Aspect 110. The method of Aspect 106, wherein the disorder of uncontrolled cellular proliferation is associated with a CK1alpha dysfunction.

Aspect 111. The method of Aspect 110, wherein the disorder of uncontrolled cellular proliferation is a cancer.

Aspect 112. The method of Aspect 111, wherein the cancer is a pediatric cancer.

Aspect 113. The method of Aspect 112, wherein the cancer is a childhood acute leukemia (AL) or a medulloblastoma (MB) cancer.

Aspect 114. The method of Aspect 111, wherein the cancer is selected from a brain cancer, lung cancer, hematological cancer, bladder cancer, colon cancer, cervical cancer, ovarian cancer, squamous cell cancer, kidney cancer, peritoneal cancer, breast cancer, gastric cancer, colorectal cancer, prostate cancer, pancreatic cancer, genitourinary tract cancer, lymphatic system cancer, stomach cancer, larynx cancer, malignant melanoma, colorectal cancer, endometrial carcinoma, thyroid cancer, rhabdosarcoma, and combinations thereof.

Aspect 115. The method of Aspect 114, wherein the cancer is selected from lung cancer, ovarian cancer, and brain cancer.

Aspect 116. The method of Aspect 115, wherein the lung cancer is selected from small-cell lung cancer, non-small cell lung cancer, and combinations thereof.

Aspect 117. The method of Aspect 115, wherein the kidney cancer is a kidney clear cell carcinoma.

Aspect 118. The method of Aspect 115, wherein the brain cancer is selected from a glioblastoma, medullablastoma, glioma, and combinations thereof.

Aspect 119. The method of Aspect 115, wherein the bladder cancer is a bladder urothelial carcinoma.

Aspect 120. The method of Aspect 115, wherein the liver cancer is a hepatic carcinoma.

Aspect 121. The method of Aspect 115, wherein the hematological cancer is selected from chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, Hodgkin lymphoma, non-Hodgkin lymphoma, and combinations thereof.

Aspect 122. The method of any one of Aspect 106-Aspect 121, further comprising the step of administering a therapeutically effective amount of at least one agent known to treat a cancer.

Aspect 123. The method of Aspect 122, wherein the at least one agent is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

Aspect 124. The method of Aspect 122, wherein the at least one agent is a DNA methyltransferase inhibitor, an HDAC-inhibitor, a glucocorticoid, an mTOR inhibitor, a cytotoxic agent, or combinations thereof.

Aspect 125. The method of Aspect 124, wherein the DNA methyltransferase inhibitor is 5-aza-2′-deoxycytidine, 5-azacytidine, zebularin, epigallocatechin-3-gallate, procaine, or combinations thereof.

Aspect 126. The method of Aspect 124, wherein the HDAC-inhibitor is vorinostat, entinostat, panbinostat, trichostatin A, mocetinostat, belinostat, dacinostat, givinostat, tubastatin A, pracinostat, droxinostat, quisinostat, romidepsin, valproic acid, AR-42 (OSU-HDAC42), tacedinaline, rocilinostat, apicidin, or combinations thereof.

Aspect 127. The method of Aspect 124, wherein the glucocorticoid is dexamethasone, prednisolone, methylprednisolone, betamethasone, triamicinolone, fludrocortisone, beclomethasone, or combinations thereof.

Aspect 128. The method of Aspect 124, wherein the mTor inhibitor is BEZ235, everolimus, temsirolimus, rapamycin, AZD8055, or combinations thereof.

Aspect 129. The method of Aspect 124, wherein the cytotoxic agent is an alkylating agent, an antimetabolite agent, an antineoplastic antibiotic agent, a mitotic inhibitor agent, a mTor inhibitor agent or other chemotherapeutic agent.

Aspect 130. The method of Aspect 129, wherein the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.

Aspect 131. The method of Aspect 129, wherein the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

Aspect 132. The method of Aspect 129, wherein the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

Aspect 133. The method of Aspect 129, wherein the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.

Aspect 134. The method of Aspect 129, wherein the mTor inhibitor is everolimus, sirolimus, temsirolimus, or combinations thereof.

Aspect 135. The method of Aspect 129, wherein the other chemotherapeutic agent is an anthracycline, cytarabine, a purine analog, sorafenib, gemtuzumab ozogamicin, rituximab, or combinations thereof.

Aspect 136. The method of Aspect 135, wherein the anthracycline is daunorubicin, idarubicin, or combinations thereof.

Aspect 137. The method of Aspect 135, wherein the purine analog is cladribine, fludarabine, clofarabine, or combinations thereof.

Aspect 138. The method of any one of Aspect 122-Aspect 137, wherein the at least one compound, the at least one pharmaceutical composition, and the at least one agent are administered sequentially.

Aspect 139. The method of any one of Aspect 122-Aspect 137, wherein the at least one compound, the at least one pharmaceutical composition, and the at least one agent are administered simultaneously.

Aspect 140. The method of any one of Aspect 122-Aspect 137, wherein the at least one compound, the at least one pharmaceutical composition, and the at least one agent are co-formulated.

Aspect 141. The method of any one of Aspect 122-Aspect 137, wherein the at least one compound, the at least one pharmaceutical composition, and the at least one agent are co-packaged.

Aspect 142. A method for modulating cereblon activity in a mammal comprising the step of administering to the mammal a therapeutically effective amount of at least one compound of any one of Aspect 1-Aspect 95, or a pharmaceutically acceptable salt thereof; or the pharmaceutical composition of any one of Aspect 96-Aspect 105.

Aspect 143. The method of Aspect 142, wherein the mammal is a human.

Aspect 144. The method of Aspect 142, wherein the mammal has been diagnosed with a need for modulating of cereblon activity prior to the administering step.

Aspect 145. The method of Aspect 142, further comprising the step of identifying a mammal in need for modulating of cereblon activity.

Aspect 146. The method of Aspect 142, wherein the modulating of cereblon activity comprises modulating the degradation of GSPT1.

Aspect 147. The method of Aspect 146, wherein the modulating the degradation of GSPT1 decreases levels of GSPT1 in a cell.

Aspect 148. The method of Aspect 142, wherein the modulating of cereblon activity comprises modulating the degradation of IKZF1.

Aspect 149. The method of Aspect 148, wherein the modulating the degradation of IKZF1 decreases levels of IKZF1 in a cell.

Aspect 150. A method for modulating GSPT1 activity in a mammal comprising the step of administering to the mammal a therapeutically effective amount of at least one compound of any one of Aspect 1-Aspect 95, or a pharmaceutically acceptable salt thereof; or the pharmaceutical composition of any one of Aspect 96-Aspect 105.

Aspect 151. The method of Aspect 143, wherein the mammal is a human.

Aspect 152. The method of Aspect 143, wherein the mammal has been diagnosed with a need for modulating GSPT1 activity prior to the administering step.

Aspect 153. The method of Aspect 143, further comprising the step of identifying a mammal in need for modulating GSPT1 activity.

Aspect 154. The method of Aspect 143, wherein the modulating the GSPT1 activity comprises modulating GSPT1 protein levels in a cell.

Aspect 155. The method of Aspect 154, wherein the modulating GSPT1 protein levels are decreased compared to a cell that is not exposed to the at least one compound or the pharmaceutical composition.

Aspect 156. A method for modulating IKZF1 activity in a mammal comprising the step of administering to the mammal a therapeutically effective amount of at least one compound of any one of Aspect 1-Aspect 95, or a pharmaceutically acceptable salt thereof; or the pharmaceutical composition of any one of Aspect 96-Aspect 105.

Aspect 157. The method of Aspect 156, wherein the mammal is a human.

Aspect 158. The method of Aspect 157, wherein the mammal has been diagnosed with a need for modulating IKZF1 activity prior to the administering step.

Aspect 159. The method of Aspect 156, further comprising the step of identifying a mammal in need for modulating IKZF1 activity.

Aspect 160. The method of Aspect 156, wherein the modulating the IKZF1 activity comprises modulating IKZF1 protein levels in a cell.

Aspect 161. The method of Aspect 160, wherein the modulating IKZF1 protein levels are decreased compared to a cell that is not exposed to the at least one compound or the pharmaceutical composition.

Aspect 162. A method for modulating of cereblon activity in at least one cell, comprising the step of contacting the at least one cell with an effective amount of at least one compound of any one of Aspect 1-Aspect 95, or a pharmaceutically acceptable salt thereof; or the pharmaceutical composition of any one of Aspect 96-Aspect 105.

Aspect 163. The method of Aspect 162, wherein the cell is mammalian.

Aspect 164. The method of Aspect 162, wherein the cell is human.

Aspect 165. The method of Aspect 162, wherein the cell has been isolated from a mammal prior to the contacting step.

Aspect 166. The method of Aspect 162, wherein contacting is via administration to a mammal.

Aspect 167. The method of Aspect 166, wherein the mammal has been diagnosed with a need for treatment of a disorder related to cereblon activity prior to the administering step.

Aspect 168. A method for modulating of GSPT1 activity in at least one cell, comprising the step of contacting the at least one cell with an effective amount of at least one compound of any one of Aspect 1-Aspect 95, or a pharmaceutically acceptable salt thereof; or the pharmaceutical composition of any one of Aspect 96-Aspect 105.

Aspect 169. The method of Aspect 162, wherein the cell is mammalian.

Aspect 170. The method of Aspect 162, wherein the cell is human.

Aspect 171. The method of Aspect 162, wherein the cell has been isolated from a mammal prior to the contacting step.

Aspect 172. The method of Aspect 162, wherein contacting is via administration to a mammal.

Aspect 173. The method of Aspect 166, wherein the mammal has been diagnosed with a need for treatment of a disorder related to cereblon activity prior to the administering step.

Aspect 174. A method for modulating of IKZF1 activity in at least one cell, comprising the step of contacting the at least one cell with an effective amount of at least one compound of any one of Aspect 1-Aspect 95, or a pharmaceutically acceptable salt thereof; or the pharmaceutical composition of any one of Aspect 96-Aspect 105.

Aspect 175. The method of Aspect 162, wherein the cell is mammalian.

Aspect 176. The method of Aspect 162, wherein the cell is human.

Aspect 177. The method of Aspect 162, wherein the cell has been isolated from a mammal prior to the contacting step.

Aspect 178. The method of Aspect 162, wherein contacting is via administration to a mammal.

Aspect 179. The method of Aspect 166, wherein the mammal has been diagnosed with a need for treatment of a disorder related to cereblon activity prior to the administering step.

Aspect 180. The method of Aspect 106, Aspect 142, Aspect 150, Aspect 156, Aspect 162, Aspect 168, or Aspect 174, wherein the compound inhibits cell proliferation with an EC50 of less than about 1 μM when determined in a cell viability assay using MV4-11 cells as described herein; and/or wherein the compound inhibits cell proliferation with an EC50 of less than about 1 μM when determined in a cell viability assay using MHHCALL4 cells as described herein; wherein the compound exhibits cereblon binding with an IC50 of less than about 10 μM using a fluorescence polarization assay as described herein; and/or wherein the compound exhibits cereblon binding with an DC50 of less than about 1 μM using a antibody-free endogenous protein detection assay (HiBit) as described herein.

Aspect 181. The method of Aspect 180, wherein the compound inhibits cell proliferation with an EC50 of less than about 0.1 μM; and/or wherein the compound exhibits cereblon binding with an IC50 of less than about 0.1 μM.

Aspect 182. The method of Aspect 180, wherein the compound inhibits cell proliferation with an EC50 of less than about 25 nM; and/or wherein the compound exhibits cereblon binding with an IC50 of less than about 25 nM.

Aspect 183. The method of Aspect 180, wherein the compound inhibits cell proliferation with an EC50 of less than about 10 nM; and/or wherein the compound exhibits cereblon binding with an IC50 of less than about 10 nM.

Aspect 184. The method of Aspect 180, wherein the compound inhibits cell proliferation with an EC50 of less than about 5 nM; and/or wherein the compound exhibits cereblon binding with an IC50 of less than about 5 nM.

Aspect 185. The method of Aspect 180, wherein the compound inhibits cell proliferation with an EC50 of less than about 1 nM; and/or wherein the compound exhibits cereblon binding with an IC50 of less than about 1 nM.

Aspect 186. A kit comprising at least one compound of any one of Aspect 1-Aspect 95, or a pharmaceutically acceptable salt thereof; and one or more of:
a) at least one agent known to increase cereblon activity;
b) at least one agent known to decrease cereblon activity;
c) at least one agent known to increase GSPT1 activity;
d) at least one agent known to decrease GSPT1 activity;
e) at least one agent known to increase IKZF1 activity;
f) at least one agent known to decrease IKZF1 activity;
g) at least one agent known to increase cellular proliferation;
h) at least one agent known to decrease cellular proliferation;
i) at least one agent known to treat a disorder associated with cereblon activity;
j) at least one agent known to treat a disorder associated with GSPT1 activity;
k) at least one agent known to treat a disorder associated with IKZF1 activity;
l) at least one agent known to treat a disorder of uncontrolled cellular proliferation; and/or
m) instructions for treating a disorder of uncontrolled cellular proliferation.

Aspect 187. The kit of Aspect 186, wherein the at least one compound or the at least one product and the at least one agent are co-formulated.

Aspect 188. The kit of Aspect 186, wherein the at least one compound or the at least one product and the at least one agent are co-packaged.

Aspect 189. The kit of any one of Aspect 186-Aspect 188, further comprising instructions to provide the compound in connection with surgery.

Aspect 190. The kit of Aspect 189, wherein the instructions provide that surgery is performed prior to the administering of at least one compound.

Aspect 191. The kit of Aspect 189, wherein the instructions provide that surgery is performed after the administering of at least one compound.

Aspect 192. The kit of Aspect 189, wherein the instructions provide that the administering of at least one compound is to effect presurgical debulking of a tumor.

Aspect 193. The kit of Aspect 189, wherein the instructions provide that surgery is performed at about the same time as the administering of at least one compound.

Aspect 194. The kit of any one of Aspect 186-Aspect 193, further comprising instructions to provide the at least one compound or the pharmaceutical composition in connection with radiotherapy.

Aspect 195. The kit of Aspect 194, wherein the instructions provide that radiotherapy is performed prior to the administering of at least one compound.

Aspect 196. The kit of Aspect 194, wherein the instructions provide that radiotherapy is performed after the step of the administering of at least one compound.

Aspect 197. The kit of Aspect 194, wherein the instructions provide that radiotherapy is performed at about the same time as the step of the administering of at least one compound.

Aspect 198. The kit of any one of Aspect 186-Aspect 197, further comprising a plurality of dosage forms, the plurality comprising one or more doses; wherein each dose comprises a therapeutically effective amount of the at least one compound or the pharmaceutical composition and the at least one agent.

Aspect 199. The kit of Aspect 198, wherein each dose of the at least one compound or the pharmaceutical composition and the at least one agent are co-formulated.

Aspect 200. The kit of Aspect 198, wherein each dose of the at least one compound or the pharmaceutical composition and the at least one agent are co-packaged.

Aspect 201. The kit of Aspect 198, wherein the dosage forms are formulated for oral administration and/or intravenous administration.

Aspect 202. The kit of Aspect 198, wherein the dosage forms are formulated for oral administration.

Aspect 203. The kit of Aspect 198, wherein the dosage forms are formulated for intravenous administration.

Aspect 204. The kit of Aspect 198, wherein the dosage form for the at least one compound or the pharmaceutical composition is formulated for oral administration and the dosage form for the at least one agent is formulated for intravenous administration.

Aspect 205. The kit of Aspect 198, wherein the dosage form for the at least one compound or the pharmaceutical composition is formulated for intravenous administration and the dosage form for the at least one agent is formulated for oral administration.

Aspect 206. Use of a compound at least one compound of any one of Aspect 1-Aspect 95, or a pharmaceutically acceptable salt thereof; or the pharmaceutical composition of any one of Aspect 96-Aspect 105; or combinations thereof in the manufacture of a medicament for the treatment of a disorder associated with a cereblon dysfunction in a mammal.

Aspect 207. Use of a compound at least one compound of any one of Aspect 1-Aspect 95, or a pharmaceutically acceptable salt thereof; or the pharmaceutical composition of any one of Aspect 96-Aspect 105; or combinations thereof in the manufacture of a medicament for the treatment of a disorder associated with a GSPT1 dysfunction in a mammal.

Aspect 208. Use of a compound at least one compound of any one of Aspect 1-Aspect 95, or a pharmaceutically acceptable salt thereof; or the pharmaceutical composition of any one of Aspect 96-Aspect 105; or combinations thereof in the manufacture of a medicament for the treatment of a disorder associated with a IKZF1 dysfunction in a mammal.

Aspect 209. Use of a compound at least one compound of any one of Aspect 1-Aspect 95, or a pharmaceutically acceptable salt thereof; or the pharmaceutical composition of any one of Aspect 96-Aspect 105; or combinations thereof in the manufacture of a medicament for the treatment of a disorder of uncontrolled cellular proliferation in a mammal.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### K. EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Cell Culture and Materials.

MV-4-11 cell line was purchased from the American Type Culture Collection (ATCC, Manassas, VA) and maintained in Iscove's Modified Dulbecco's medium (IMDM; (ATCC) supplemented with 10% FBS. MHHCALL4 cell line was purchased from the German Collection of Microorganisms and Cell Cultures GmbH (DSMZ) and maintained in RPMI 1640 medium supplemented with 10% fetal bovine serum (HyClone), penicillin/streptomycin (100 U/mL), and glutamine (100 mM) at 37° C. in a humidified 5% C02 incubator. Exponentially growing MV-4-11 cells were plated at 1250 cells per well and MHH-CALL-4 cells were plated at 7,500 cells per well in Corning 8804BC white 384-well assay plates and incubated overnight at 37° C. in a humidified 5% CO₂ incubator. Compounds were transferred to the assay plate from a dose-response plate using a Pintool on a Biomek FXP Laboratory Automation Workstation (Beckman Coulter). Cytotoxicity was determined following 72 hours of incubation using Promega Cell Titer Glo reagent according to the manufacturer's recommendation. Luminescence was measured on an Envision plate reader (Perkin-Elmer).

### 2. Cell Proliferation Assay.

Compounds were screened in human cancer cell lines and each cell line was cultured in the complete medium recommended by the vendor and seeded in Corning 8804BC white 384-well assay plates at density of 1250 cells per well for MV-4-11 cells and of 7,500 cells per well for MHH-CALL-4 cells. After overnight incubation at 37° C. in a humidified 5% CO₂ incubator, cells were treated with compounds in dose-response format using a Pintool on a Biomek FXP Laboratory Automation Workstation (Beckman Coulter). After 72 h incubation, cell proliferation was assessed using Cell Titer-Gbo (CTG) luminescent cell viability assay (Promega) according to the manufacturer's instruction. Luminescence signal was measured using an Envision plate reader (Perkin-Elmer).

3. hGSPT1 HiBiT Assay.

HEK293 hGSPT1 HiBiT tag cells were seeded in white 384-well assay plates at density of 800 cells per well in triplicates. After overnight incubation, cells were treated with compounds in dose-response format using a Pintool on a Biomek FXP Laboratory Automation Workstation (Beckman Coulter). After 4 hrs incubation, the level of GSPT1 HiBiT tag protein was evaluated using Nano-Glo® HiBiT Lytic Detection System (Promega) according to the manufacturer's instruction. Luminescence signal was measured using an Envision plate reader (Perkin-Elmer).

### 4. CRBN Fluorescence Polarization Assay.

In this competitive fluorescent polarization assay Cy5 conjugated lenalidomide analog (Cy5-O-Len)¹³ was used as a fluorescent probe. 6×His-CRBN-DDB1 protein (200 nM) and Cy5-O-Len probe (30 nM) were combined in 20 mM HEPES pH 7, 150 mM NaCl, 0.005% Tween-20 assay buffer. 20 μL of this assay cocktail was dispensed into wells of Corning 3821 black 384-well plates. Compounds were transferred to the assay plate from a dose-response plate using a Pintool on a Biomek FXP Laboratory Automation Workstation (Beckman Coulter). The plates were incubated in the dark for 1 hour at room temperature and then read on an Envision plate reader (PerkinElmer, Massachusetts, USA). IC₅₀ values were determined using a proprietary software RISE (Robust Investigation of Screening Experiments), developed in house on the Pipeline Pilot platform (Biovia, v. 17.2.0). Data represent the mean of three independent determinations.

### 5. Chemistry: General Methods and Synthesis.

All reagents and solvents were obtained from commercially available sources and were used without further purification. Reactions were set up in air and carried out under nitrogen atmosphere. Parallel synthesis was accomplished using MiniBlock XT synthesizers purchased from Mettler-Toledo AutoChem placed on a stirring hot plate. Library compounds were filtered using a 96 well Thomson 2 mL filter plate (25 μm) containing Celite 545 into a Waters 96 well 2 ml receiving plate prior to purification. Automated weighing was done using a Bohdan Balance Automator (Mettler-Toldeo AutoChem) and parallel evaporations were carried out using a Genevac HT-24. HPLC analyses were accomplished using an UPLC/UV/ELSD/SQD (Single Quadrupole Detector) with stationary phase: BEH C18, 1.7 μm, solvents: A: 0.1% formic acid in water, B: 0.1% formic acid in acetonitrile, detector types: PDA (210 to 400 nm) and ELSD. Library purification was performed on the Waters purification/analytical LC/UV/ELSD system. Column: Gemini Aixia Packed C18 50 mm×30 mm, 5 μm. Collection: UV at 214 nm and/or ELSD. Gradients: water/acetonitrile/0.1% formic acid, beginning acetonitrile percentage varied based on retention times determined in pre-purification analysis on the UPLC. Nuclear magnetic resonance (NMR) spectra were obtained on a Bruker NMR spectrometer at 500 MHz for 1H-NMR spectra and 125 MHz for 13C-NMR spectra. Chemical shifts (ppm) are reported relative to the solvent peak. Signals are designated as follows: s, singlet; d, doublet; dd, doublet of doublet; t, triplet; q, quadruplet; m, multiplet. Coupling constants (J) are expressed in Hertz. High resolution mass spectral data were obtained on a Waters Xevo G2 QTof mass spectrometer. The purity of final compounds was performed on an Acquity UPLC BEH C18 1.7 μm, 2.1×50 mm column (Waters Corporation, Milford, MA) using an Acquity ultra performance liquid chromatography system. The flow rate was 0.7 mL/min. The sample injection volume was 3 μL. The UPLC column was maintained at 50° C. and the gradient program started at 90% A (0.1% formic acid in MilliQ H2O), changed to 95% B (0.1% formic acid in Acetonitrile) over 2.5 min, held for 0.35 minutes, then to 90% A over 0.05 minutes.

### 6. General Procedure for the Synthesis of Disclosed Compounds.

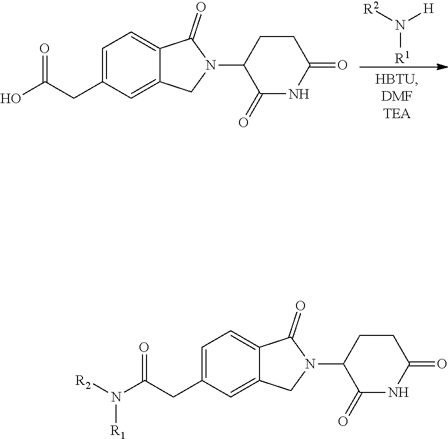

Briefly, in a 48-position Mettler Toledo XT reaction block containing 11.5×110 mm test tubes with stir bars was added 1.3 equiv. of HBTU to each reaction tube, followed by 1 equiv. of 2-(2-(2,6-dioxopiperidin-3-yl)-1-oxoisoindolin-5-yl)acetic acid and the appropriate amines (1.2 equiv.). 350 μL of DMF was added, followed by 5 equiv. of TEA to each tube. The inert manifold was placed on the XT-block and the reaction block was stirred under house nitrogen overnight at room temperature. The reaction progress was monitored by UPLC. Each reaction vessel was diluted with DMSO to −1 ml and then filtered through a 96-well Thomson filter plate (containing a small amount of Celite 545) and attached to a Waters 96 deep well collection plate. The plate was briefly centrifuged to aid the filtrations and the Celite was washed with an additional 200 ul of DMSO. Prepurification analysis in the UPLC was done with water/acetonitrile/0.1% formic acid. Library purification was performed on the Waters purification/analytical LC/UV/ELSD system and parallel evaporations were carried out using a TurboVap® LV evaporator.

### 7. Representative Disclosed Compounds.

The following representative disclosed compounds (see Table 1 below) were prepared by the general procedure described herein above.

TABLE 1
Representative Disclosed Compounds

| Cmpd | |
|---|---|
| No. | Structure |
| 1 | 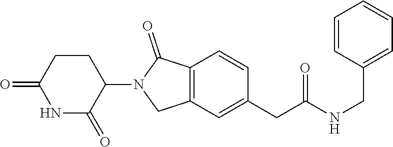 [SMILES: O=C(Cc1ccc2c(c1)CN(C1CCC(=O)NC1=O)C2=O)NCc1ccccc1] |
| 2 | 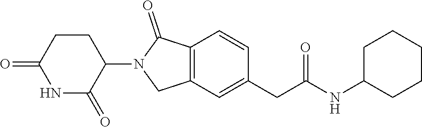 [SMILES: O=C1CCC(N2Cc3cc(CC(=O)NC4CCCCC4)ccc3C2=O)C(=O)N1] |
| 3 | 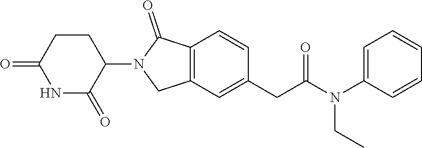 [SMILES: CCN(C(=O)Cc1ccc2c(c1)CN(C1CCC(=O)NC1=O)C2=O)c1ccccc1] |
| 4 | 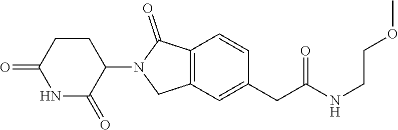 [SMILES: COCCNC(=O)Cc1ccc2c(c1)CN(C1CCC(=O)NC1=O)C2=O] |
| 5 | 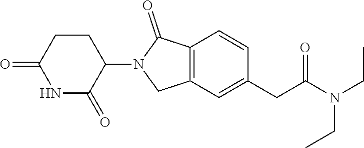 [SMILES: CCN(CC)C(=O)Cc1ccc2c(c1)CN(C1CCC(=O)NC1=O)C2=O] |
| 6 | 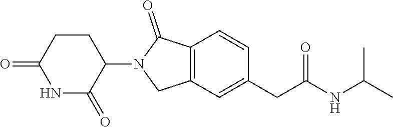 [SMILES: CC(C)NC(=O)Cc1ccc2c(c1)CN(C1CCC(=O)NC1=O)C2=O] |
| 7 | 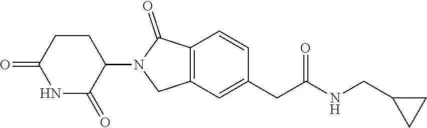 [SMILES: O=C(Cc1ccc2c(c1)CN(C1CCC(=O)NC1=O)C2=O)NCC1CC1] |
| 8 | 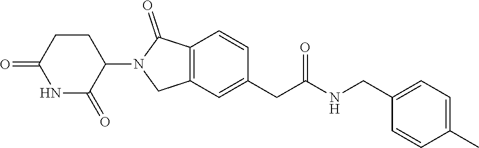 [SMILES: Cc1ccc(CNC(=O)Cc2ccc3c(c2)CN(C2CCC(=O)NC2=O)C3=O)cc1] |
| 9 | 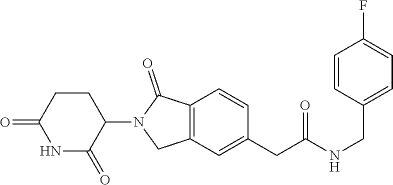 [SMILES: O=C(Cc1ccc2c(c1)CN(C1CCC(=O)NC1=O)C2=O)NCc1ccc(F)cc1] |
| 10 | 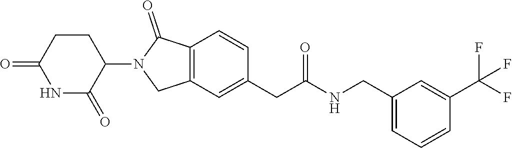 [SMILES: O=C(Cc1ccc2c(c1)CN(C1CCC(=O)NC1=O)C2=O)NCc1cccc(C(F)(F)F)c1] |
| 11 | 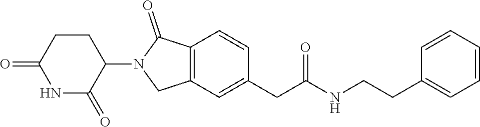 [SMILES: O=C(Cc1ccc2c(c1)CN(C1CCC(=O)NC1=O)C2=O)NCCc1ccccc1] |
| 12 | 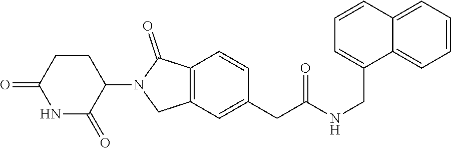 [SMILES: O=C(Cc1ccc2c(c1)CN(C1CCC(=O)NC1=O)C2=O)NCc1cccc2ccccc12] |
| 13 | 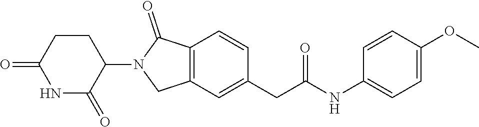 [SMILES: COc1ccc(NC(=O)Cc2ccc3c(c2)CN(C2CCC(=O)NC2=O)C3=O)cc1] |
| 14 | 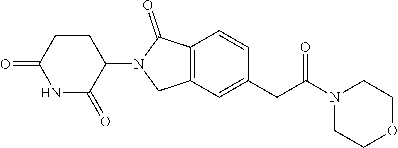 [SMILES: O=C1CCC(N2Cc3cc(CC(=O)N4CCOCC4)ccc3C2=O)C(=O)N1] |
| 15 | 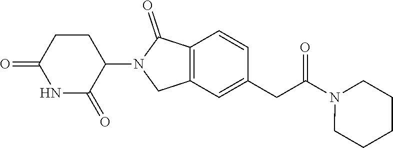 [SMILES: O=C1CCC(N2Cc3cc(CC(=O)N4CCCCC4)ccc3C2=O)C(=O)N1] |
| 16 | 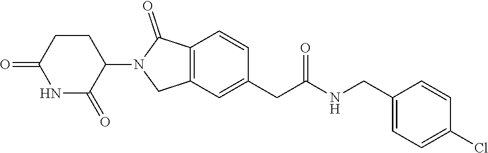 [SMILES: O=C(Cc1ccc2c(c1)CN(C1CCC(=O)NC1=O)C2=O)NCc1ccc(Cl)cc1] |
| 17 | 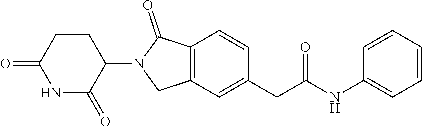 [SMILES: O=C1CCC(N2Cc3cc(CC(=O)Nc4ccccc4)ccc3C2=O)C(=O)N1] |
| 18 | 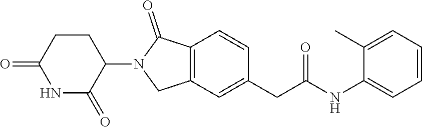 [SMILES: Cc1ccccc1NC(=O)Cc1ccc2c(c1)CN(C1CCC(=O)NC1=O)C2=O] |
| 19 | 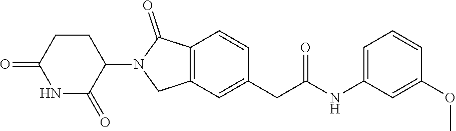 [SMILES: COc1cccc(NC(=O)Cc2ccc3c(c2)CN(C2CCC(=O)NC2=O)C3=O)c1] |
| 20 | 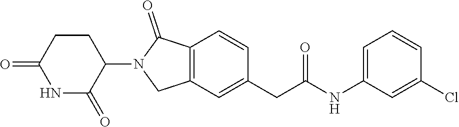 [SMILES: O=C1CCC(N2Cc3cc(CC(=O)Nc4cccc(Cl)c4)ccc3C2=O)C(=O)N1] |
| 21 | 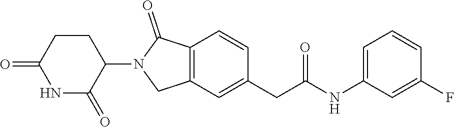 [SMILES: O=C1CCC(N2Cc3cc(CC(=O)Nc4cccc(F)c4)ccc3C2=O)C(=O)N1] |
| 22 | 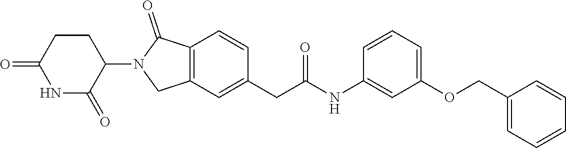 [SMILES: O=C1CCC(N2Cc3cc(CC(=O)Nc4cccc(OCc5ccccc5)c4)ccc3C2=O)C(=O)N1] |
| 23 | 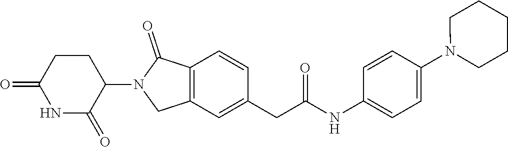 [SMILES: O=C1CCC(N2Cc3cc(CC(=O)Nc4ccc(N5CCCCC5)cc4)ccc3C2=O)C(=O)N1] |

TABLE 1
Representative Disclosed Compounds, continued.

| | | | | | Retention | |
|---|---|---|---|---|---|---|
| Cmpd | Amount | | | | Time | LCMS |
| No. | (mg) | Yield | Purity | MW | (min) | (M + 1) |
| 1 | 4.3 | 14.6% | >95% | 391.4 | 0.97 | 392.0 |
| 2 | 7.3 | 25.4% | >95% | 383.4 | 1.02 | 384.0 |
| 3 | 46 | 151.0% | >95% | 405.5 | 1.19 | 406.1 |
| 4 | 7.7 | 28.6% | >95% | 359.4 | 0.6 | 359.9 |
| 5 | 26.4 | 98.0% | >95% | 357.4 | 0.89 | 357.9 |
| 6 | 8.5 | 33.0% | >95% | 343.4 | 0.72 | 344.0 |
| 7 | 19 | 71.3% | >95% | 355.4 | 0.78 | 356.0 |
| 8 | 7.8 | 25.7% | >95% | 405.5 | 1.08 | 406.0 |
| 9 | 5.3 | 17.3% | >95% | 409.4 | 1.02 | 410.0 |
| 10 | 7.6 | 22.1% | >95% | 459.4 | 1.22 | 460.0 |
| 11 | 8.1 | 26.6% | >95% | 405.5 | 1.02 | 406.0 |
| 12 | 7.8 | 23.6% | >95% | 441.5 | 1.22 | 442.1 |
| 13 | 6.3 | 20.6% | >95% | 407.4 | 0.98 | 408.1 |
| 14 | 16.1 | 57.8% | >95% | 371.4 | 0.62 | 372.0 |
| 15 | 40.9 | 148.0% | >95% | 369.4 | 0.92 | 370.1 |
| 16 | 6.6 | 20.7% | >95% | 425.9 | 1.13 | 426.1 |
| 17 | 5.8 | 20.5% | >95% | 377.4 | 1.01 | 378.0 |
| 18 | 5.6 | 19.1% | >95% | 391.4 | 1.01 | 392.0 |
| 19 | 6.8 | 22.3% | >95% | 407.4 | 1.05 | 408.1 |
| 20 | 9.6 | 31.1% | >95% | 411.8 | 1.24 | 412.1 |
| 21 | 10.3 | 34.7% | >95% | 395.4 | 1.08 | 396.0 |
| 22 | 8 | 22.1% | >95% | 483.5 | 1.49 | 484.2 |
| 23 | 35.5 | 103.0% | >95% | 460.5 | 0.63 | 460.9 |

### 8. Results—Activity of Disclosed Compounds.

The GSPT1 HiBit assay and cell proliferation activity was determined as described herein above, and the results are provided below in Table 2 for representative disclosed compounds. Compound number (“Cmpd No.”) refers to the compound number (and associated compound information such as structure, name, etc.) as provided in Table 1.

TABLE 2
Activity for representative compounds.

| | GSPT1 | MHHCALL4 Cell | MV411 Cell |
|---|---|---|---|
| Cmpd | HiBit Assay | Proliferation Assay | Proliferation Assay |
| No. | (DC₅₀, μM) | (EC₅₀, μM) | (EC₅₀, μM) |
| 1 | <0.001 | <0.001 | <0.001 |
| 2 | >7.1 | cytostatic at 10 nM | >5.8 |
| 3 | — | >10.7 | >8.7 |
| 4 | — | >8.2 | >6.7 |
| 5 | — | — | >1.73 |
| 6 | — | >8.5 | >6.9 |
| 7 | — | >10.1 | >8.2 |
| 8 | <0.001 | <0.001 | <0.001 |
| 9 | <0.001 | <0.001 | 0.003 |
| 10 | <0.001 | <0.001 | <0.001 |
| 11 | <0.001 | <0.001 | — |
| 12 | <0.001 | <0.001 | 0.005 |
| 13 | <0.001 | <0.001 | <0.001 |
| 14 | >2.7 | 0.021 | >2.2 |
| 15 | — | >10.7 | >8.7 |
| 16 | <0.001 | <0.001 | — |
| 17 | >8.1 | 0.004 | 0.744 |
| 18 | >5.97 | 0.002 | >4.9 |
| 19 | — | <0.001 | 0.005 |
| 20 | <0.001 | <0.001 | 0.005 |
| 21 | >3.11 | <0.001 | >4.5 |
| 22 | <0.001 | <0.001 | <0.001 |
| 23 | <0.001 | <0.001 | 0.002 |

* “—” indicates that the assay was not carried out.

### 9. Prospective Pharmacokinetics (Pk) Study.

Healthy female 001 mice (8-12 weeks old) weighing between 20 to 30 g are procured from Global, India. Three mice are housed in each cage. Temperature and humidity are maintained at 22±3 00 and 30-70%, respectively and illumination is controlled to give a sequence of 12 hr light and 12 hr dark cycle. Temperature and humidity are recorded by auto-controlled data logger system. All the animals are provided laboratory rodent diet (Envigo Research private Ltd, Hyderabad). Reverse osmosis water treated with ultraviolet light is provided ad libitum. Formulation vehicle: 5% v/v NMP: 5% v/v Solute HS-15 and 90% v/v normal saline. Animals in Group 1 are administered intravenously as slow bolus injection through tail vein, with solution formulation of a representative disclosed compound at 3 mg/kg dose. Animals in Group 2 are administered through oral route with solution formulation of the representative disclosed compound at 10 mg/kg dose. The dosing volume for intravenous is 5 mL/kg and for oral administration 10 mL/kg. Blood samples (approximately 60 μL) are collected under light isoflurane anesthesia (Surgivet®) from retro orbital plexus from a set of three mice at 0.08 (for IV only), 0.25, 0.5, 1, 2, 4, 6 (for P0 only), 8, 12 and 24 hr. Immediately after blood collection, plasma is harvested by centrifugation at 4000 rpm, 10 min at 40° C. and samples were stored at −70±10° C. until bioanalysis. Concentrations of the representative disclosed compound in mouse plasma samples were determined by fit for purpose LC-MS/MS method. Non-Compartmental-Analysis tool of Phoenix WinNonlin® (Version 8.0) can be used to assess the pharmacokinetic parameters. Peak plasma concentration (Cmax) and time for the peak plasma concentration (Tmax) are observed values. The areas under the concentration time curve (AUClast and AUCinf) can be calculated by linear trapezoidal rule.

The terminal elimination rate constant, kₑ, can be determined by regression analysis of the linear terminal portion of the log plasma concentration-time curve. Clearance can be estimated as Dose/AUC_{inf} and Vss as CL*MRT. The oral bioavailability can be calculated as ratio of dose normalized AUC of oral and intravenous group multiplied by 100.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A compound having a structure represented by a formula: 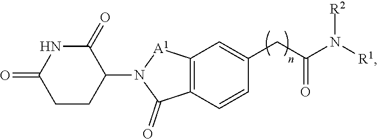
wherein each of A¹ is selected from —(C═O)— and —(CH₂)—;
wherein n is selected from 1, 2, and 3;
wherein R¹ is selected from —(C0-C3 alkanediyl)-phenyl and —(C0-C3 alkanediyl)-naphthyl; and
wherein R¹ is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰;
wherein -A¹⁰ is selected from substituted naphthyl, substituted phenyl, substituted cycloalkyl, and substituted heterocycloalkyl; and
wherein -A²⁰- is selected from —(CH₂)ₓ—, —(O—CH₂)ₓ—, —(NH—CH₂)ₓ—, —(CH₂—O)ₓ—, and —(CH₂—NH)ₓ—;
wherein x is selected from 0, 1, 2, and 3;
wherein R² is selected from hydrogen, substituted C1-C10 alkyl, and substituted C1-C10 heteroalkyl;
or a pharmaceutically acceptable salt thereof.

2. The compound of claim 1, wherein A¹ is —(CH₂)—.

3. The compound of claim 1, wherein n is 1 or 2.

4. The compound of claim 1, wherein n is 1.

5. The compound of claim 1, wherein R¹ is —(CH₂)ₘ-phenyl; and wherein the phenyl is optionally substituted with 0, 1, or 2 groups independently selected from halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰; and wherein m is selected from 0, 1, 2, and 3.

6. The compound of claim 5, wherein m is 0.

7. The compound of claim 5, wherein the phenyl has a structure represented by a formula: 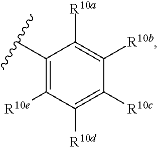
wherein each of R^{10a}, R^{10b}, R^{10c}, R^{10d}, and R^{10e} is independently selected from hydrogen, halogen, —SF₅, —CN, —N₃, —NH₂, —OH, —CN, —SCF₃, C1-C3 alkoxy, C1-C3 haloalkyl, C1-C3 aminoalkyl, C1-C3 alkylamino, C1-C3 hydroxyalkyl, —O—(C1-C3 haloalkyl), C3-C8 cycloalkyl, C1-C6 alkyl, -A²⁰-A¹⁰, and -A¹⁰.

8. The compound of claim 7, wherein the compound has a structure represented by a formula:

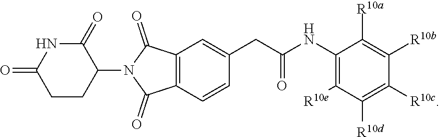

9. The compound of claim 7, wherein the compound has a structure represented by a formula:

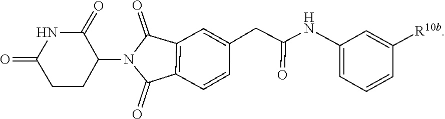

10. The compound of claim 7, wherein the compound has a structure represented by a formula:

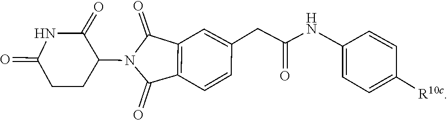

11. The compound of claim 7, wherein the compound has a structure represented by a formula:

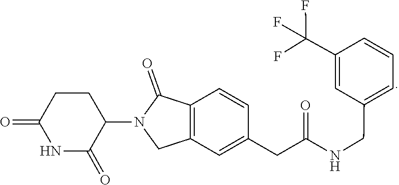 [SMILES: O=C(Cc1ccc2c(c1)CN(C1CCC(=O)NC1=O)C2=O)NCc1cccc(C(F)(F)F)c1]

12. A pharmaceutical composition comprising a therapeutically effective amount of a compound of claim 1, or a pharmaceutically acceptable salt, solvate, or polymorph thereof, and a pharmaceutically acceptable carrier.

13. A method for the treatment of a disorder of uncontrolled cellular proliferation in a mammal comprising the step of administering to the mammal a therapeutically effective amount of at least one compound of claim 1, or a pharmaceutically acceptable salt thereof; or a pharmaceutical composition thereof.

14. The method of claim 13, wherein the disorder of uncontrolled cellular proliferation is associated with a CK1alpha dysfunction.

15. The method of claim 14, wherein the disorder of uncontrolled cellular proliferation is a cancer.

16. A method for for-modulating GSPT1 activity in a mammal comprising administering to the mammal a therapeutically effective amount of at least one compound of claim 1, or a pharmaceutically acceptable salt thereof; or a pharmaceutical composition thereof.

17. A method for for-modulating IKZF1 activity in a mammal comprising administering to the mammal a therapeutically effective amount of at least one compound of claim 1, or a pharmaceutically acceptable salt thereof; or a pharmaceutical composition thereof.

18. A method for for-modulating cereblon activity in a mammal comprising administering to the mammal a therapeutically effective amount of at least one compound of claim 1, or a pharmaceutically acceptable salt thereof; or a pharmaceutical composition thereof.

19. A kit comprising at least one compound of claim 1, or a pharmaceutically acceptable salt thereof; and one or more of:
a) at least one agent known to increase cereblon activity;
b) at least one agent known to decrease cereblon activity;
c) at least one agent known to increase GSPT1 activity;
d) at least one agent known to decrease GSPT1 activity;
e) at least one agent known to increase IKZF1 activity;
f) at least one agent known to decrease IKZF1 activity;
g) at least one agent known to increase cellular proliferation;
h) at least one agent known to decrease cellular proliferation;
i) at least one agent known to treat a disorder associated with cereblon activity;
j) at least one agent known to treat a disorder associated with GSPT1 activity;
k) at least one agent known to treat a disorder associated with IKZF1 activity;
l) at least one agent known to treat a disorder of uncontrolled cellular proliferation; and/or instructions for treating a disorder of uncontrolled cellular proliferation.

20. (canceled)
